(19) United States
(12) Patent Application Publication
(10) Pub. No.: US 2024/0226131 A1
(43) Pub. Date: Jul. 11, 2024

(54) 2'-CHLORO-2'-FLUORO-N2-AMINO-N6-METHYLAMINO PURINE NUCLEOTIDES FOR FLAVIVIRUS TREATMENT

(71) Applicant: Atea Pharmaceuticals, Inc., Boston, MA (US)

(72) Inventors: Jean-Pierre Sommadossi, Boston, MA (US); Adel Moussa, Burlington, MA (US)

(73) Assignee: Atea Pharmaceuticals, Inc., Boston, MA (US)

(21) Appl. No.: 18/592,037

(22) Filed: Feb. 29, 2024

Related U.S. Application Data
(63) Continuation of application No. PCT/US2022/042545, filed on Sep. 2, 2022.
(60) Provisional application No. 63/240,578, filed on Sep. 3, 2021.

(51) Int. Cl. A61K 31/7076; A61P 31/14

(52) U.S. Cl. CPC A61K 31/7076; A61P 31/14

(57) ABSTRACT

The present invention is directed to new methods for the treatment of an infection by a virus from the genus Flavivirus, particularly Dengue fever virus, Yellow Fever virus, Zika virus, and West Nile virus, in a host in need thereof, typically a human.

### CROSS-REFERENCE TO RELATED APPLICATIONS

This application is a continuation of International Patent Application No. PCT/US2022/042545, filed in the U.S. Receiving Office on Sep. 2, 2022, which claims the benefit of U.S. Provisional Application No. 63/240,578, which was filed Sep. 3, 2021. The entirety of each of these applications is hereby incorporated by reference for all purposes.

### FIELD OF THE INVENTION

The present invention is directed to treatments for an infection caused by a virus from the genus Flavivirus, including dengue virus, yellow fever virus, Zika virus, and West Nile virus, in a host in need thereof, typically a human.

### BACKGROUND OF THE INVENTION

Flaviviruses are a genus of vector borne viruses which have a positive-sense single stranded RNA genome. The genome of a positive sense RNA virus can be directly translated into viral proteins without intermediate transcription steps or need for viral polymerases in the virion.

Infections caused by viruses of the genus Flavivirus include but are not limited to dengue fever, West Nile fever, yellow fever, Zika virus disease, Kyasanur Forest disease, Powassan disease, Wesselsbron disease, Rio bravo, Rocio, Negishi, and the encephalitises such as California encephalitis, central European encephalitis, Ilheus virus, Murray Valley encephalitis, St. Louis encephalitis, Japanese B encephalitis, Louping ill, and Russian spring-rodents summer encephalitis.

Dengue fever is one of the most prevalent Flavivirus diseases. There is currently no approved therapeutic treatment other than palliative care. A number of clinical trials have been carried out to evaluate treatments for dengue fever but have failed to meet their primary efficacy end points (Low et al., The Journal of Infectious Diseases, 2017 Mar. 1; 215 (Suppl 2) S96-S102). While a preventative vaccine has been developed, its efficacy varies with the age of the recipient and serotype of the infection (Hadinegoro et al. New England Journal of Medicine, 2015; 373:1195-206; Halstead et al. Vaccine 2016; 34:1643-1647; Biering et al. Nature News and Views, 2021, 598, 420-421). In 2021, Janssen Pharmaceuticals initiated clinical trials of a new therapeutic for dengue fever (JNJ-A07). This compound is an inhibitor of the NS3/NS4B viral enzymes (see for example, Kaptein, S. J. F. et al. A pan-serotype dengue virus inhibitor targeting the NS3-NS4B interaction. Nature, 2021, 598, 504-509). Currently, the primary recourse is supportive care for those infected, which entails treatment of the symptoms, including high fever, headache, severe joint and muscle pain, and nausea. Fluid replacement and analgesics, along with acetaminophen, aspirin, and nonsteroidal anti-inflammatory drugs are sometimes effective. Without treatment, the mortality rate of dengue fever can be as high as 30%.

There are also no approved drugs for an infection caused by West Nile virus (West Nile fever), another virus of the Flavivirus genus. Physicians typically recommend intensive support therapy, which may involve hospitalization, intravenous fluids, use of a ventilator to assist breathing, medications to control seizures, brain swelling, nausea and vomiting, as well as the use of antibiotics to prevent secondary bacterial infections.

The medical state of the art is similar for Zika virus disease. There is no vaccine or specific therapeutic treatment available. The focus is on relieving symptoms, including rest, rehydration and acetaminophen for fever and pain.

There is a vaccine for yellow fever. Yellow Fever Vaccine (YF-Vax), manufactured by Sanofi Pasteur, is recommended for persons aged nine and older who are traveling to areas of high risk, including South America and Africa. Prevention is the only specific option available for yellow fever, as no anti-viral treatment exists. As is the case in many Flavivirus infections, an emphasis is placed on relieving symptoms by easing fever, muscle pain, and dehydration. This palliative care is complicated by the risk of internal bleeding, so typical fever reducers and pain relievers like aspirin and nonsteroidal anti-inflammatory drugs are not recommended.

Atea Pharmaceuticals, Inc. has disclosed 2′-methyl-2′-fluoro-N²-amino-N⁶-methylamino purine nucleotide phosphoramidates and pharmaceutically acceptable salts thereof for HCV treatment (U.S. Pat. Nos. 9,828,410; 10,239,911; 10,000,523; 10,815,266; 10,870,672; 10,870,673; 10,875,885; and 10,005,811). Atea disclosed the hemisulfate salt of 2′-methyl-2′-fluoro-N²-amino-N⁶-methylamino purine nucleotide phosphoramidates in U.S. Pat. Nos. 10,519,186; 10,906,928; and 10,894,804. Atea has also disclosed 2′-substituted and disubstituted-N²-amino-N⁶-substituted purine nucleotides for positive strand RNA virus treatment, including Flaviviruses such as dengue virus, West Nile virus, and yellow fever virus (for example, U.S. Pat. No. 10,946,033). Atea reports highly active compounds against COVID-19 in U.S. Pat. No. 10,874,687.

United States Patent publications and PCT applications that describe 2′-disubstituted-nucleotide polymerase inhibitors for the treatment of Flaviviridae include those filed by Idenix Pharmaceuticals (WO2013177219; WO2015081297; and WO2015081133), Gilead Sciences (WO2012012465), Emory University (WO2015164812; WO2017165489; and WO2015038596), University College Cardiff Consultants Limited (WO2014076490) and Medivir AB (US20150175648; WO2015034420; and US20180036330).

The need for Flavivirus treatments is increasing as Flaviviruses are expected to continue spreading into uninfected areas of the world and mutate under drug pressure. The medical need is particularly strong for a safe, effective, and well-tolerated anti-viral treatment, as higher viremia levels are associated with more severe disease. Further, additional treatments are necessary due to the anticipated need for combination therapies to avoid drug resistance.

It is therefore an object of the present invention to provide new treatments and pharmaceutical compositions to treat Flavivirus infections.

### SUMMARY OF THE INVENTION

The present invention provides treatments for Flavivirus infections in a host in need thereof, typically a human, comprising administering an effective amount of a 2′-chloro-2′-fluoro-N²-amino-N⁶-methylamino purine nucleotide phosphoramidate or a pharmaceutical acceptable salt thereof as described herein.

The invention includes a compound of Formula I or Formula II which are 2′-chloro-2′-fluoro-N²-amino-N⁶-methylamino purine nucleotide phosphoramidate, or their pharmaceutically acceptable salts, is advantageous for treatment of viruses from the genus Flavivirus, when administered in an effective amount to a host, typically a human, in need thereof. The host can alternatively be any animal that carries the Flavivirus infection.

In particular, the nucleotide phosphoramidates of Formulas I and nucleotides of Formula II demonstrate advantageous activity, for example, against dengue virus, West Nile virus, Zika virus and yellow fever virus. In specific embodiments, methods are provided to treat dengue virus or yellow fever virus that comprise administering to a host in need thereof, particularly a human, a 2′-chloro-2′-fluoro-N²-amino-N⁶-methylamino purine nucleotide phosphoramidate or pharmaceutically acceptable salt thereof as described herein. In a principal embodiment, the nucleotide is a phosphoramidate. In certain embodiments, the nucleotide is a stabilized phosphate prodrug.

Thus, in one embodiment, the invention is a method for treating an infection of a Flavivirus in a host in need thereof, for example a human, comprising administering an effective amount of a compound of Formula I. In one embodiment, a method is presented to treat a host, including a human, infected with a Flavivirus with an effective amount of a compound of Formula I. In certain embodiments, a method is presented to treat a host, including a human, infected with dengue virus, Zika virus, West Nile virus, or yellow fever, with an effective amount of a compound of Formula I:

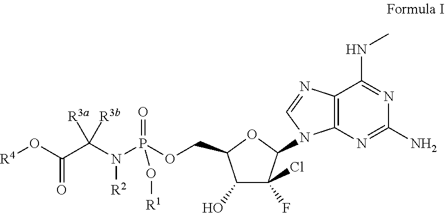

wherein:
R¹ is hydrogen, optionally substituted C₁₋₆alkyl (including methyl, ethyl, propyl, and isopropyl), optionally substituted C₃₋₇cycloalkyl, or aryl (including phenyl and naphthyl) and in certain embodiments, R¹ is optionally substituted —(C₁-C₄alkyl)aryl, optionally substituted heteroaryl, or optionally substituted heteroalkyl;
R² is hydrogen or optionally substituted C₁₋₆alkyl (including methyl, ethyl, propyl, and isopropyl);
R^{3a} and R^{3b} are independently selected from hydrogen, C₁₋₆alkyl (including methyl, ethyl, propyl, and isopropyl), and optionally substituted C₃₋₇cycloalkyl; and
R⁴ is hydrogen, optionally substituted C₁₋₆alkyl (including methyl, ethyl, propyl, and isopropyl), optionally substituted C₁₋₆haloalkyl, or optionally substituted C₃₋₇cycloalkyl and in another embodiment, R⁴ is optionally substituted —(C₁-C₄alkyl)aryl (for example, benzyl), optionally substituted aryl, optionally substituted heteroaryl, or optionally substituted heteroalkyl; or a pharmaceutically acceptable salt thereof. In certain nonlimiting embodiments, the salt is a hemisulfate salt.

In certain embodiments, an N⁶-methylamino-purine compound used in the present invention is metabolized to a 5′-monophosphate of the N⁶-methylamino-purine, and then subsequently metabolized at the 6-position to generate an active guanine triphosphate compound in a manner that provides good activity and therapeutic index.

As an example, the metabolism of the 2′-chloro-2′-fluoro-N²-amino-N⁶-methylamino purine nucleotide phosphoramidate involves metabolism of the phosphoramidate to the 5′-monophosphate and subsequent metabolism of the N²-amino-N⁶-methylamino purine base to generate the 2′-chloro-2′-fluoro guanine nucleoside 5′-monophosphate. The monophosphate is then anabolized to the active species which is the 5′-triphosphate (Scheme 1).

In particular, it has been discovered that a 5′-stabilized nucleotide phosphate prodrug (i.e., a derivative that can be metabolized to the mono-, di-, or tri-5′-phosphate nucleotide) of a 2′-chloro-2′-fluoro-N²-amino-N⁶-methylamino purine nucleotide, as well as other 2′-chloro-2′-fluoro-N²-amino-N⁶-methylamino purine nucleotides as described below, are active against viruses in the genus Flavivirus. For example, as discussed in Example 5 and shown in Table 1, Compound 8 is potent against dengue virus (EC_{50=0.32} μM), West Nile virus (EC₅₀=0.32 μM), and yellow fever virus (EC₅₀=0.12 μM).

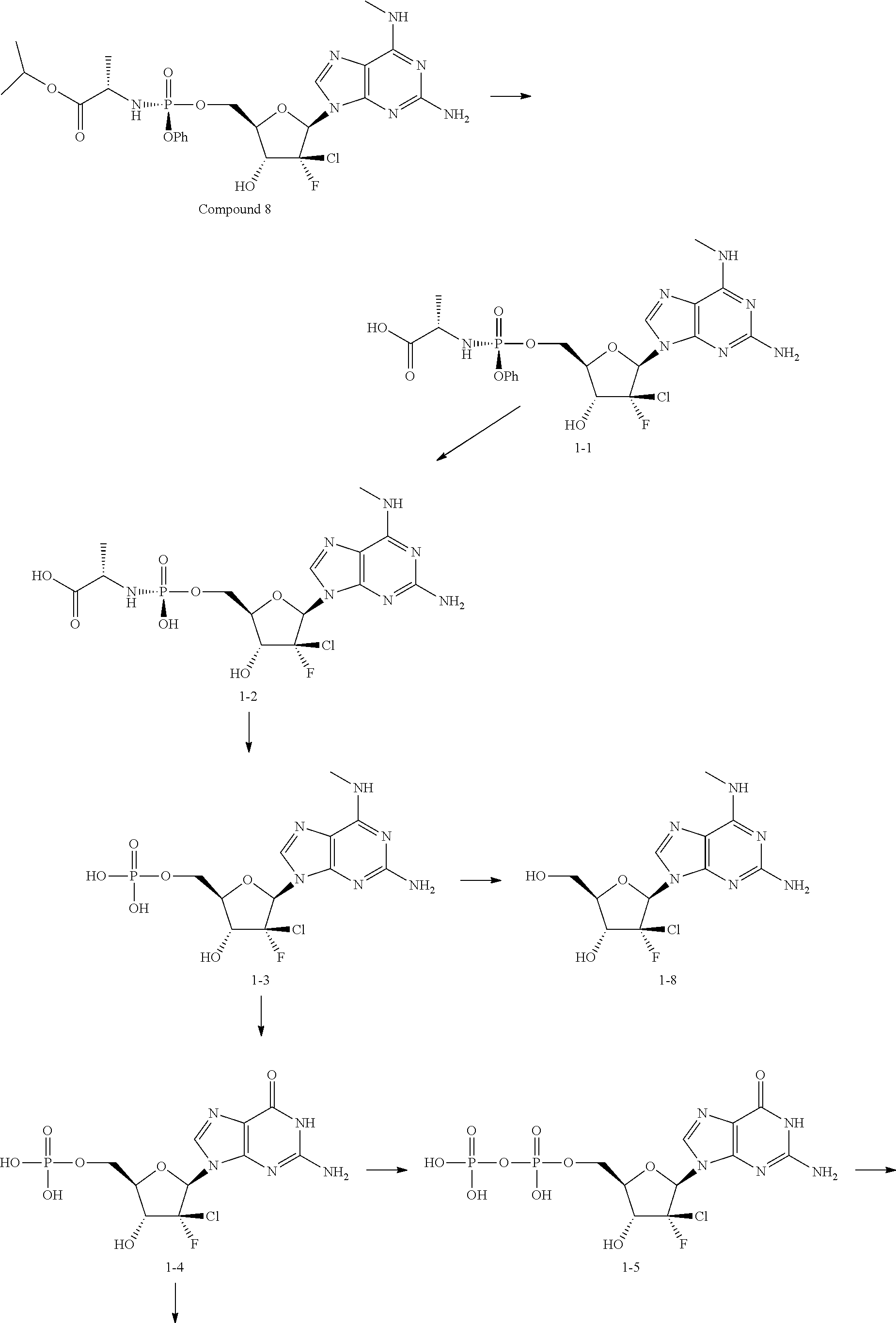

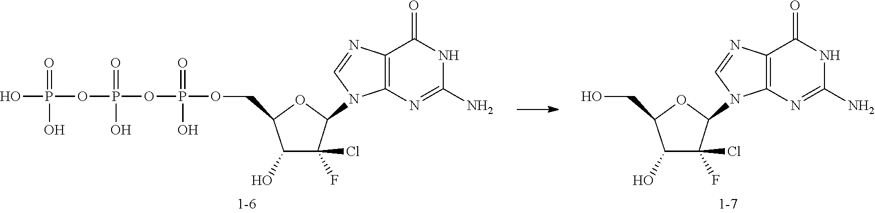 [SMILES: Nc1nc2c(ncn2[C@@H]2O[C@H](COP(=O)(O)OP(=O)(O)OP(=O)(O)O)[C@@H](O)[C@]2(F)Cl)c(=O)[nH]1 ; Nc1nc2c(ncn2[C@@H]2O[C@H](CO)[C@@H](O)[C@]2(F)Cl)c(=O)[nH]1]

The present invention also includes the use of a compound of Formula II wherein R⁵ is a monophosphate, a diphosphate, a triphosphate, or R^{10A}, wherein R^{10A} is a stabilized phosphate prodrug that metabolizes in vivo to a monophosphate, diphosphate, or triphosphate to treat or prevent infections of a Flavivirus, in particular dengue virus, in a host in need thereof as described herein:

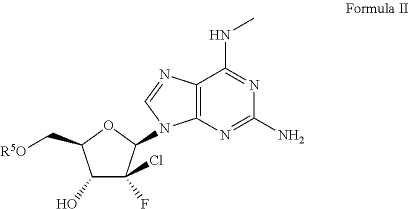

wherein:
R⁵ is selected from

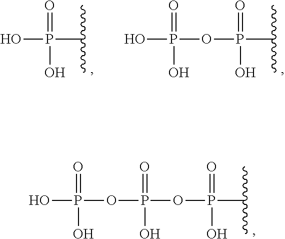 [SMILES: *P(=O)(O)O ; *P(=O)(O)OP(=O)(O)O ; *P(=O)(O)OP(=O)(O)OP(=O)(O)O]

and R^{10A},
   R^{10A} is a stabilized phosphate prodrug that metabolizes in vivo to a monophosphate, diphosphate, or triphosphate;
or a pharmaceutically acceptable salt thereof, and
all other variables are as previously defined herein.

In certain embodiments, R⁵ is selected from

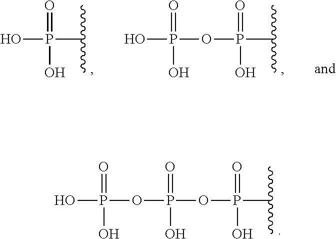 [SMILES: *P(=O)(O)O ; *P(=O)(O)OP(=O)(O)O ; *P(=O)(O)OP(=O)(O)OP(=O)(O)O]

Unless otherwise specified, a compound described herein is provided in the β-D-configuration. Likewise, when in phosphoramidate or thiophosphoramidate form, the amino acid portion can be in the L- or D-configuration. In certain embodiments, a compound can be provided in a β-L-configuration. Likewise, any substituent group that exhibits chirality can be provided in racemic, enantiomeric, diastereomeric form or any mixture thereof. Where a phosphoramidate, thiophosphoramidate or other stabilized phosphorus prodrug in which the phosphorus exhibits chirality is used as the R⁵ stabilized phosphate prodrug, it can be provided as an R or S chiral phosphorus derivative or a mixture thereof, including a racemic mixture. All of the combinations of these stereoconfigurations are included in the invention described herein.

The present invention includes the use of a compound of Formula I or Formula II or a pharmaceutically acceptable composition, salt, or prodrug thereof, as described herein in an effective amount to treat a Flavivirus, for example, dengue virus.

Methods, uses, and pharmaceutical compositions are provided for the treatment of a host, for example a human, infected with a Flavivirus via administration of an effective amount of the compound or its pharmaceutically acceptable salt.

A compound or formulation that includes a compound can also be administered in an effective amount prophylactically to prevent or minimize the progression of clinical illness in individuals who are Flavivirus antibody- or antigen-positive.

The invention also includes a method of treatment or prophylaxis of a Flavivirus, including drug resistant and multidrug resistant forms of a Flavivirus and related disease states, conditions, or complications of a Flavivirus infection, as well as other conditions that are secondary to a Flavivirus infection, such as weakness, loss of appetite, weight loss, breast enlargement (especially in men), rash (especially on the palms), difficulty with clotting of blood, spider-like blood vessels on the skin, confusion, coma (encephalopathy), buildup of fluid in the abdominal cavity (ascites), esophageal varices, portal hypertension, kidney failure, enlarged spleen, decrease in blood cells, anemia, thrombocytopenia, jaundice, and hepatocellular cancer, among others. The method comprises administering to a host in need thereof an effective amount of at least one 2′-chloro-2′-fluoro-N²-amino-N⁶-methylamino purine nucleotide phosphoramidate as described herein, optionally in combination with at least one additional therapeutic agent, for example, an additional anti-Flavivirus agent, further in combination with a pharmaceutically acceptable carrier, additive, and/or excipient.

The phosphorus in any of the Formulas above may be chiral and thus can be provided as an R or S enantiomer or mixture thereof, including a racemic mixture.

Non-limiting embodiments include:

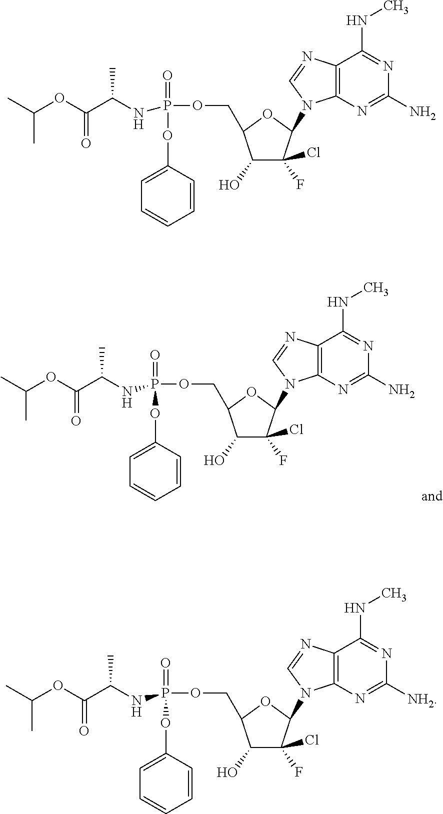 [SMILES: CNc1nc(N)nc2c1ncn2[C@@H]1OC(COP(=O)(N[C@@H](C)C(=O)OC(C)C)Oc2ccccc2)[C@@H](O)[C@]1(F)Cl ; CNc1nc(N)nc2c1ncn2[C@@H]1OC(CO[P@@](=O)(N[C@@H](C)C(=O)OC(C)C)Oc2ccccc2)[C@@H](O)[C@]1(F)Cl ; "and" ; CNc1nc(N)nc2c1ncn2[C@@H]1OC(CO[P@](=O)(N[C@@H](C)C(=O)OC(C)C)Oc2ccccc2)[C@@H](O)[C@]1(F)Cl]

In some embodiments, methods, uses, and compositions are provided for the treatment of a host in need thereof infected with a Flavivirus, described herein, for example, dengue virus, Zika virus, West Nile virus, or yellow fever virus. For example, a method of the invention can comprise administration of an effective amount of a compound of Formula I alone or in combination with another anti-Flavivirus viral agent to treat the infected host in need thereof. In certain embodiments, it is useful to administer a combination of drugs that modulate the same or a different pathway or inhibit a different target in the virus. As the disclosed 2′-chloro-2′-fluoro-N²-amino-N⁶-methylamino nucleotides are polymerase inhibitors, it can be advantageous to administer an effective amount of the compound to a host in need thereof in combination with an effective amount of a protease inhibitor or an NS5 inhibitor. The invention can also be used in combination with administration of an effective amount of a structurally different polymerase inhibitor such as another compound described herein or otherwise known to those in the art. The invention can also be used in combination with administration of an effective amount of ribavirin and/or interferon. The invention can for example be used in combination with administration of an effective amount of an inhibitor of the NS3/NS4B interaction, such as but not limited to JNJ-A07.

The 2′-chloro-2′-fluoro-N²-amino-N⁶-methylamino purine nucleotide phosphoramidates described for use in the invention are typically administered orally, for example in pill or tablet form, but may be administered via another route which the attending physician considers appropriate, including via intravenous, inhalation, systemic, transdermal, subcutaneous, topical, parenteral, or other suitable route.

### DETAILED DESCRIPTION OF THE INVENTION

The invention describes methods, uses, and compositions as described herein for the treatment of Flavivirus infections, or exposure to a Flavivirus, in humans or another host animal that includes the administration of an effective amount of a compound of Formula I or Formula II, as described herein, or a pharmaceutically acceptable salt or prodrug thereof, optionally in a pharmaceutically acceptable carrier. A compound used herein either possesses anti-Flavivirus activity or is metabolized to a compound that exhibits such activity. In certain embodiments, a treatment is presented that includes the administration of a compound of Formula I or Formula II, or a pharmaceutically acceptable salt thereof for an infection of dengue virus, Zika virus, West Nile virus, or yellow fever virus, in a host in need thereof, including a human.

A compound or composition can also be used to treat conditions related to or occurring as a result of a Flavivirus viral exposure. In certain embodiments, the present invention can also be used prophylactically to prevent or retard the progression of clinical illness in individuals who are Flavivirus antibody- or Flavivirus antigen-positive.

In particular, it has been discovered that 5′-stabilized phosphate prodrugs or derivatives of a 2′-chloro-2′-fluoro purine nucleoside, such as a 2′-chloro-2′-fluoro-N²-amino-N⁶-methylamino purine nucleotide phosphoramidate as described below, are highly active against Flaviviruses, for example dengue virus, West Nile virus, Zika virus, or yellow fever virus.

Unless otherwise specified, a compound described herein is provided in the β-D-configuration. In certain embodiments, a compound can be provided in a β-L-configuration. Likewise, any substituent group that exhibits chirality can be provided in racemic, enantiomeric, diastereomeric form or any mixture thereof. Where a phosphoramidate, thiophosphoramidate or other stabilized phosphorus prodrug in which the phosphorus exhibits chirality is used as the R⁵ stabilized phosphate prodrug, it can be provided as an R or S chiral phosphorus derivative or a mixture thereof, including a racemic mixture. The amino acid of the phosphoramidate or thiophosphoramidate can be in the D- or L-configuration, or a mixture thereof, including a racemic mixture. All of the combinations of these stereo configurations are included in the invention described herein.

The present invention includes the following features:
(a) A method for treatment or prophylaxis of a Flavivirus infection that includes the administration of an effective amount of Formulas I or II and pharmaceutically acceptable salts and prodrugs thereof and as described herein;
(b) Use of an effective amount of Formulas I or II, and pharmaceutically acceptable salts and prodrugs thereof in the manufacture of a medicament for treatment of a Flavivirus infection;
(c) A method for manufacturing a medicament intended for the therapeutic use for treating a Flavivirus infection, characterized in that an effective amount of a Formula I or II as described herein is used in the manufacture; and
(d) A pharmaceutical formulation comprising an effective host-treating amount of the Formulas I or II or a pharmaceutically acceptable salt thereof together with a pharmaceutically acceptable carrier or diluent to treat a Flavivirus;
(e) A compound of Formula I or II for use to treat a Flavivirus infection; and
(f) Any one of embodiments (a)-(e) wherein the Flavivirus is selected from the group consisting of dengue virus, yellow fever virus, West Nile virus, and Zika virus.

### I. 2′-Chloro-2′-Fluoro-N²-Amino-N⁶-Methylamino Purine Nucleotide Phosphoramidates Used in the Invention

An active compound of the invention is one depicted, for example, in Formula I, which can be provided in a pharmaceutically acceptable composition, salt or stabilized phosphate prodrug thereof:

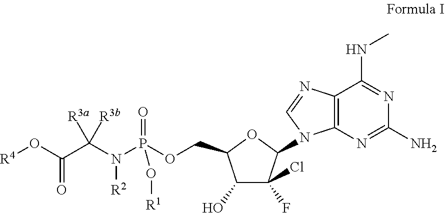

wherein:
R¹ is hydrogen, optionally substituted C₁₋₆alkyl (including methyl, ethyl, propyl, and isopropyl), optionally substituted C₃₋₇cycloalkyl, or optionally substituted aryl (including phenyl and naphthyl) and in certain embodiments, R¹ is optionally substituted —(C₁-C₄alkyl)aryl (for example, benzyl), optionally substituted heteroaryl, or optionally substituted heteroalkyl;
R² is hydrogen or optionally substituted C₁₋₆alkyl (including methyl, ethyl, propyl, and isopropyl);
R^{3a} and R^{3b} are independently selected from hydrogen, optionally substituted C₁₋₆alkyl (including methyl, ethyl, propyl, and isopropyl), and optionally substituted C₃₋₇cycloalkyl;
R⁴ is hydrogen, optionally substituted C₁₋₆alkyl (including methyl, ethyl, propyl, and isopropyl), optionally substituted C₁₋₆haloalkyl, or optionally substituted C₃₋₇cycloalkyl and in an certain embodiments, R⁴ is optionally substituted —(C₁-C₄alkyl)aryl, optionally substituted aryl, optionally substituted heteroaryl, or optionally substituted heteroalkyl; and

In some embodiments of Formula I, R¹ is phenyl.

In some embodiments of Formula I, R¹ is naphthyl.

In some embodiments of Formula I, R² is hydrogen.

In some embodiments of Formula I, R² is methyl.

In some embodiments of Formula I, R^{3a} is hydrogen and R^{4b} is methyl.

In some embodiments of Formula I, R^{3a} is hydrogen and R^{4b} is ethyl.

In some embodiments of Formula I, R^{3a} is hydrogen and R^{4b} is n-propyl.

In some embodiments of Formula I, R^{3a} is hydrogen and R^{4b} is isopropyl.

In some embodiments of Formula I, R⁴ is methyl.

In some embodiments of Formula I, R⁴ is ethyl.

In some embodiments of Formula I, R⁴ is n-propyl.

In some embodiments of Formula I, R⁴ is isopropyl.

In some embodiments of Formula I, the compound is the Sₚ-isomer and the phosphoramidate is in the L-configuration.

In some embodiments of Formula I, the compound is the Rₚ-isomer and the phosphoramidate is in the L-configuration.

In some embodiments of Formula I or II, the pharmaceutically acceptable salt is the hemi-sulfate salt.

In a typical embodiment, the compound is a β-D isomer with reference to the corresponding nucleoside (i.e., in the naturally occurring configuration). In certain embodiments, the compound is provided as a β-L isomer. The compound is typically at least 90% free of the opposite enantiomer, and can be at least 95%, 96%, 97%, 98%, 99% or even 100% free of the opposite enantiomer. Unless described otherwise, the compound is at least 90% free of the opposite enantiomer.

The metabolism of the 2′-chloro-2′-fluoro-N²-amino-N⁶-methylamino purine nucleotide phosphoramidate results in the production of the corresponding 5′-monophosphate. Subsequent metabolism of the N2-amino-N⁶-methylamino purine base generates the 2′-chloro-2′-fluoro guanine nucleoside as the 5′-monophosphate. The 5′-monophosphate is then further anabolized to the 5′-triphosphate, which is the active species. The metabolic pathway for the 2′-chloro-2′-fluoro-N²-amino-N⁶-methylamino purine nucleotide phosphoramidate is illustrated in Scheme 1.

### ILLUSTRATIVE EMBODIMENTS OF THE PRESENT INVENTION

In certain nonlimiting embodiments the present invention includes:

1. A method comprising administering an effective amount of a compound of Formula I:

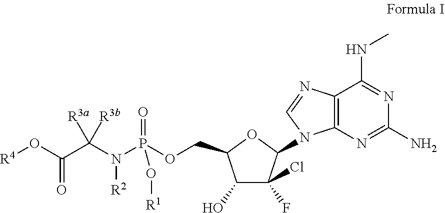

to treat a human host in need thereof infected with a Flavivirus;
wherein:
R¹ is hydrogen, C₁₋₆alkyl, C₃₋₇cycloalkyl, aryl, —(C₁-C₄alkyl)aryl, heteroaryl, or heteroalkyl;
R² is hydrogen or C₁₋₆alkyl;
R^{3a} and R^{3b} are independently selected from hydrogen, C₁₋₆alkyl, and C₃₋₇cycloalkyl; and
R⁴ is hydrogen, C₁₋₆alkyl, C₁₋₆haloalkyl, C₃₋₇cycloalkyl, —(C₁-C₄alkyl)aryl, aryl, heteroaryl, or heteroalkyl;
   or a pharmaceutically acceptable salt thereof.

2. The method of embodiment 1, wherein R¹ is hydrogen.

3. The method of embodiment 1, wherein R¹ is phenyl.

4. The method of any one of embodiments 1-3, wherein R² is hydrogen.

5. The method of any one of embodiments 1-4, wherein R^{3a} and R^{3b} are hydrogen and C₁₋₆alkyl.

6. The method of any one of embodiments 1-5 wherein R⁴ is C₁₋₆ alkyl.

7. The method of embodiment 1,
wherein
R¹ is aryl;
R² is hydrogen;
R^{3a} is methyl; and
R⁴ is C₁₋₆alkyl.

8. The method of embodiment 7 wherein the compound is of formula:

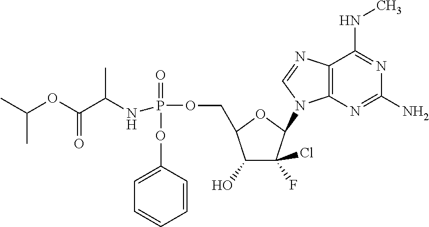 [SMILES: CNc1nc(N)nc2c1ncn2[C@@H]1OC(COP(=O)(NC(C)C(=O)OC(C)C)Oc2ccccc2)[C@@H](O)[C@]1(F)Cl]

or a pharmaceutically acceptable salt thereof.

9. The method of embodiment 8 wherein the compound is selected from:

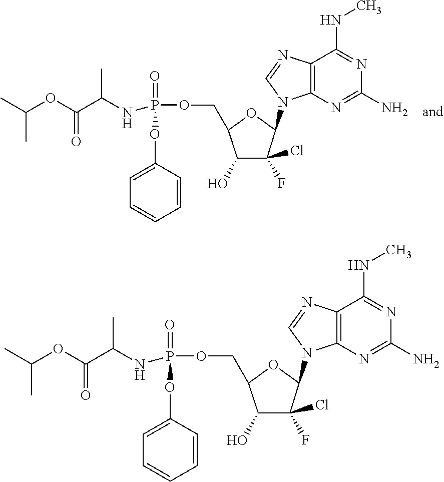 [SMILES: CNc1nc(N)nc2c1ncn2[C@@H]1OC(CO[P@](=O)(NC(C)C(=O)OC(C)C)Oc2ccccc2)[C@@H](O)[C@]1(F)Cl ; "and" ; CNc1nc(N)nc2c1ncn2[C@@H]1OC(CO[P@@](=O)(NC(C)C(=O)OC(C)C)Oc2ccccc2)[C@@H](O)[C@]1(F)Cl]

or a pharmaceutically acceptable salt thereof.

10. The method of embodiment 8 wherein the compound is:

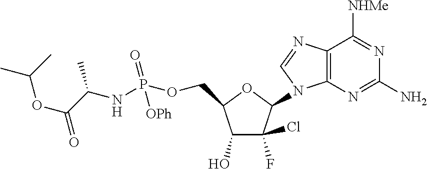 [SMILES: CNc1nc(N)nc2c1ncn2[C@@H]1O[C@H](COP(=O)(N[C@@H](C)C(=O)OC(C)C)Oc2ccccc2)[C@@H](O)[C@]1(F)Cl]

or a pharmaceutically acceptable salt thereof.

11. The method of embodiment 8 wherein the compound is:

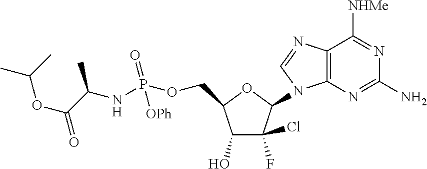 [SMILES: CNc1nc(N)nc2c1ncn2[C@@H]1O[C@H](COP(=O)(N[C@H](C)C(=O)OC(C)C)Oc2ccccc2)[C@@H](O)[C@]1(F)Cl]

or a pharmaceutically acceptable salt thereof.

12. The method of embodiment 9 wherein the compound is:

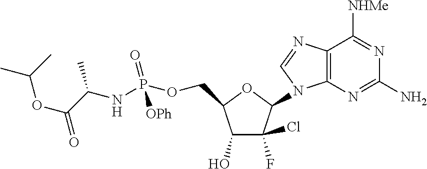 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@]1(F)Cl]

or a pharmaceutically acceptable salt thereof.

13. The method of embodiment 9, wherein the compound is:

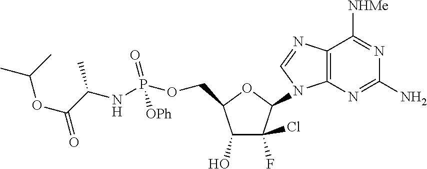 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@]1(F)Cl]

or a pharmaceutically acceptable salt thereof.

14. The method of embodiment 9 wherein the compound is:

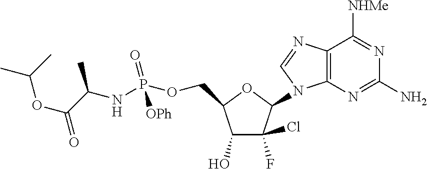 [SMILES: CNc1nc(N)nc2c1ncn2[C@@H]1O[C@H](CO[P@@](=O)(N[C@H](C)C(=O)OC(C)C)Oc2ccccc2)[C@@H](O)[C@]1(F)Cl]

or a pharmaceutically acceptable salt thereof.

15. The method of embodiment 9, wherein the compound is:

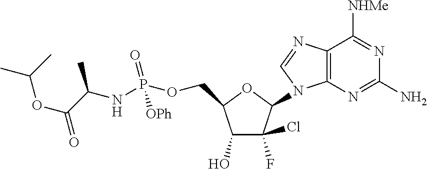 [SMILES: CNc1nc(N)nc2c1ncn2[C@@H]1O[C@H](CO[P@](=O)(N[C@H](C)C(=O)OC(C)C)Oc2ccccc2)[C@@H](O)[C@]1(F)Cl]

or a pharmaceutically acceptable salt thereof.
16. The method of any one of embodiments 1-15, wherein the pharmaceutically acceptable salt is the hemisulfate.

17 The method of embodiment 8 wherein the compound is:

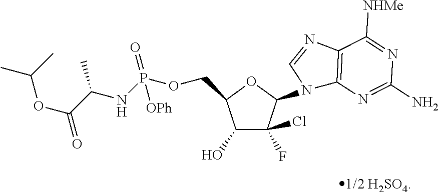 [SMILES: CNc1nc(N)nc2c1ncn2[C@@H]1O[C@H](COP(=O)(N[C@@H](C)C(=O)OC(C)C)Oc2ccccc2)[C@@H](O)[C@]1(F)Cl]

18. The method of embodiment 8 wherein the compound is:

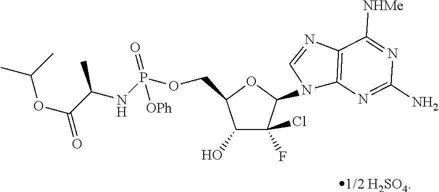 [SMILES: CNc1nc(N)nc2c1ncn2[C@@H]1O[C@H](COP(=O)(N[C@H](C)C(=O)OC(C)C)Oc2ccccc2)[C@@H](O)[C@]1(F)Cl]

19. The method of embodiment 9 wherein the compound is:

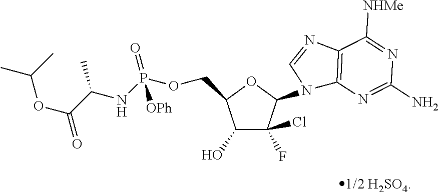 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@]1(F)Cl]

20. The method of embodiment 9, wherein the compound is:

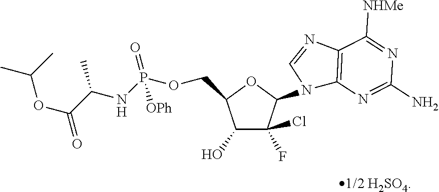 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@]1(F)Cl]

21. The method of embodiment 9 wherein the compound is:

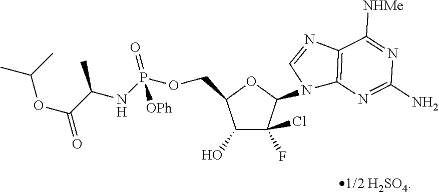 [SMILES: CNc1nc(N)nc2c1ncn2[C@@H]1O[C@H](CO[P@@](=O)(N[C@H](C)C(=O)OC(C)C)Oc2ccccc2)[C@@H](O)[C@]1(F)Cl]

22. The method of embodiment 9, wherein the compound is:

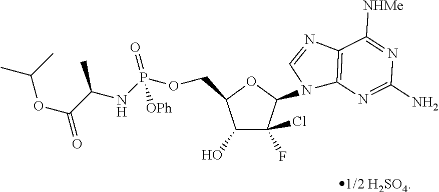 [SMILES: CNc1nc(N)nc2c1ncn2[C@@H]1O[C@H](CO[P@](=O)(N[C@H](C)C(=O)OC(C)C)Oc2ccccc2)[C@@H](O)[C@]1(F)Cl]

23. A method comprising administering an effective amount of a compound of Formula II:

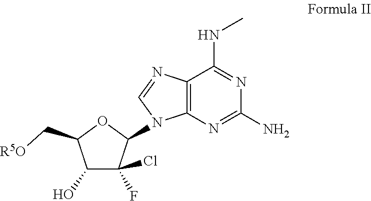

to treat a human host in need thereof infected with a Flavivirus;
   wherein:
R⁵ is selected from

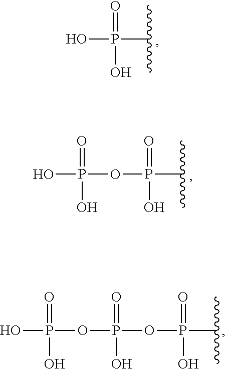 [SMILES: *P(=O)(O)O ; *P(=O)(O)OP(=O)(O)O ; *P(=O)(O)OP(=O)(O)OP(=O)(O)O]

and R^{10A}; and
R^{10A} is a stabilized phosphate prodrug that metabolizes in vivo to a monophosphate, diphosphate, or triphosphate;
   or a pharmaceutically acceptable salt thereof.

24. The method of embodiment 23, wherein R⁵ is R^{10a}

25. The method of any of embodiments 1-24, wherein the virus is selected from dengue virus, West Nile virus, yellow fever virus, and Zika virus.

26. The method of embodiment 25, wherein the virus is dengue virus.

27. The method of embodiment 25, wherein the virus is yellow fever virus.

28. The method of embodiment 25, wherein the virus is West Nile virus.

29. The method of embodiment 25, wherein the virus is Zika virus.

30. The method of any one of embodiments 1-29, wherein the compound is in a dosage form suitable for oral administration.

31. The method of embodiment 30, wherein the oral dosage form is a solid oral dosage form.

32. The method of embodiment 31, wherein the oral dosage form is a tablet.

33. The method of embodiment 31, wherein the oral dosage form is a capsule.

34. The method of any one of embodiments 1-33, wherein from about 500 mg to about 850 mg of the compound is administered.

35. The method of any one of embodiments 1-33, wherein from about 500 mg to about 650 mg of the compound is administered.

36. The method of any one of embodiments 1-33, wherein from about 600 mg to about 750 mg of the compound is administered.

37. The method of any one of embodiments 1-33, wherein from about 650 mg to about 850 mg of the compound is administered.

38. The method of any one of embodiments 1-33, wherein at least about 550 mg of the compound is administered.

39. The method of any one of embodiments 1-33, wherein at least about 575 mg of the compound is administered.

40. The method of any one of embodiments 1-33, wherein at least about 600 mg of the hemisulfate salt of the compound is administered.

41. The method of any one of embodiments 1-33, wherein at least about 625 mg of the hemisulfate salt of the compound is administered.

42. The method of any one of embodiments 1-33, wherein at least about 700 mg of the compound is administered.

43. The method of any one of embodiments 1-33, wherein at least about 775 mg of the hemisulfate salt of the compound is administered.

44. The method of any one of embodiments 1-43, wherein the compound is administered once per day.

45. The method of any one of embodiments 1-43, wherein the compound is administered twice per day.

46. The method of any one of embodiments 1-43, wherein the compound is administered four times per day.

47. A compound of Formula I:

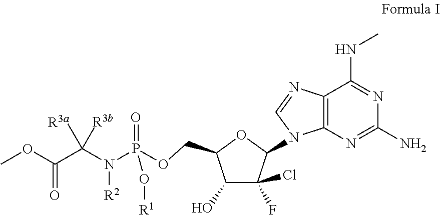

for use to treat a human host in need thereof infected with a Flavivirus;
wherein:
R¹ is hydrogen, C₁₋₆alkyl, C₃₋₇cycloalkyl, aryl, —(C₁-C₄alkyl)aryl, heteroaryl, or heteroalkyl;
R² is hydrogen or C₁₋₆alkyl;
R^{3a} and R^{3b} are independently selected from hydrogen, C₁₋₆alkyl, and C₃₋₇cycloalkyl; and
R⁴ is hydrogen, C₁₋₆alkyl, C₁₋₆haloalkyl, C₃₋₇cycloalkyl, —(C₁-C₄alkyl)aryl, aryl, heteroaryl, or heteroalkyl;
   or a pharmaceutically acceptable salt thereof.

48. The compound for use of embodiment 47, wherein R¹ is hydrogen.

49. The compound for use of embodiment 47, wherein R¹ is phenyl.

50. The compound for use of any one of embodiments 47-49, wherein R² is hydrogen.

51. The compound for use of any one of embodiments 47-50, wherein R^{3a} and R^{3b} are hydrogen and C₁₋₆alkyl.

52. The compound for use of any one of embodiments 47-51 wherein R⁴ is C₁₋₆alkyl.

53. The compound for use of embodiment 47,
wherein
R¹ is aryl;
R² is hydrogen;
R^{3a} is methyl; and
R⁴ is C₁₋₆alkyl.

54. The compound for use of embodiment 47 wherein the compound is of formula:

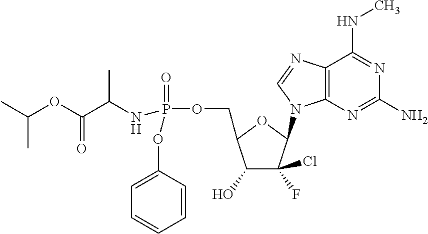 [SMILES: CNc1nc(N)nc2c1ncn2[C@@H]1OC(COP(=O)(NC(C)C(=O)OC(C)C)Oc2ccccc2)[C@@H](O)[C@]1(F)Cl]

or a pharmaceutically acceptable salt thereof.

55. The compound for use of embodiment 54 wherein the compound is selected from:

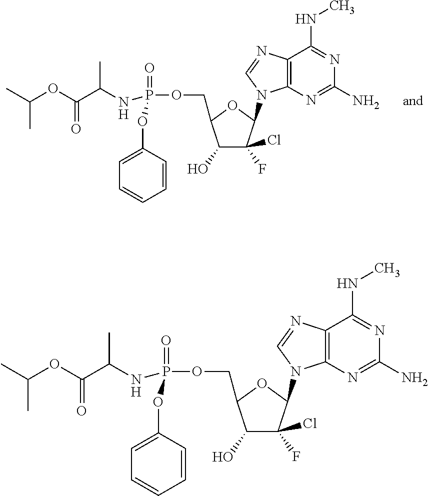 [SMILES: CNc1nc(N)nc2c1ncn2[C@@H]1OC(CO[P@](=O)(NC(C)C(=O)OC(C)C)Oc2ccccc2)[C@@H](O)[C@]1(F)Cl ; CNc1nc(N)nc2c1ncn2[C@@H]1OC(CO[P@@](=O)(NC(C)C(=O)OC(C)C)Oc2ccccc2)[C@@H](O)[C@]1(F)Cl]

or a pharmaceutically acceptable salt thereof.

56. The compound for use of embodiment 54 wherein the compound is:

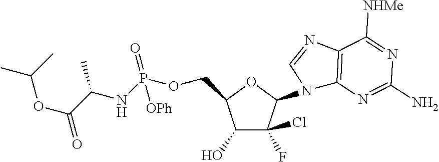 [SMILES: CNc1nc(N)nc2c1ncn2[C@@H]1O[C@H](COP(=O)(N[C@@H](C)C(=O)OC(C)C)Oc2ccccc2)[C@@H](O)[C@]1(F)Cl]

or a pharmaceutically acceptable salt thereof.

57. The compound for use of embodiment 54 wherein the compound is:

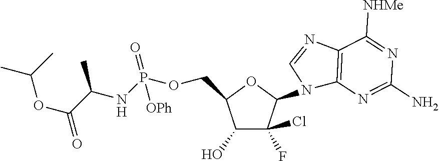 [SMILES: CNc1nc(N)nc2c1ncn2[C@@H]1O[C@H](COP(=O)(N[C@H](C)C(=O)OC(C)C)Oc2ccccc2)[C@@H](O)[C@]1(F)Cl]

or a pharmaceutically acceptable salt thereof.

58. The compound for use of embodiment 56 wherein the compound is:

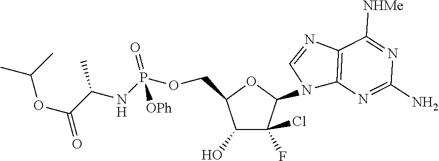 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@]1(F)Cl]

or a pharmaceutically acceptable salt thereof.

59. The compound for use of embodiment 56, wherein the compound is:

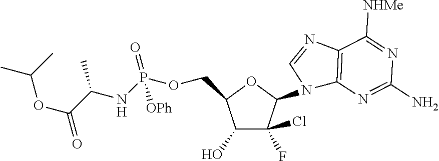 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@]1(F)Cl]

or a pharmaceutically acceptable salt thereof.

60. The compound for use of embodiment 57 wherein the compound is:

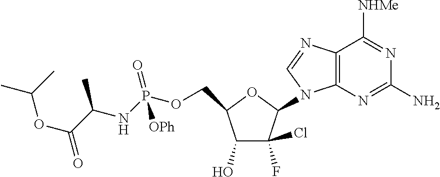 [SMILES: CNc1nc(N)nc2c1ncn2[C@@H]1O[C@H](CO[P@@](=O)(N[C@H](C)C(=O)OC(C)C)Oc2ccccc2)[C@@H](O)[C@]1(F)Cl]

or a pharmaceutically acceptable salt thereof.

61. The compound for use of embodiment 57, wherein the compound is:

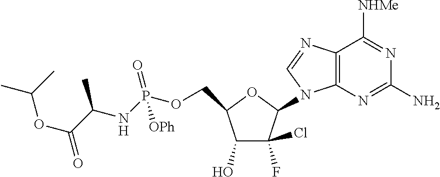 [SMILES: CNc1nc(N)nc2c1ncn2[C@@H]1O[C@H](CO[P@](=O)(N[C@H](C)C(=O)OC(C)C)Oc2ccccc2)[C@@H](O)[C@]1(F)Cl]

or a pharmaceutically acceptable salt thereof.

62. The compound for use of any one of embodiments 47-61, wherein the pharmaceutically acceptable salt is the hemisulfate.

63. The compound for use of embodiment 56 wherein the compound is:

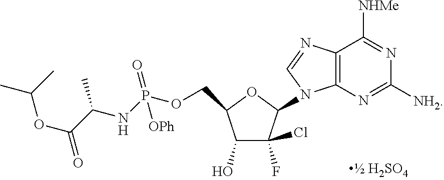 [SMILES: CNc1nc(N)nc2c1ncn2[C@@H]1O[C@H](COP(=O)(N[C@@H](C)C(=O)OC(C)C)Oc2ccccc2)[C@@H](O)[C@]1(F)Cl]

64. The compound for use of embodiment 57 wherein the compound is:

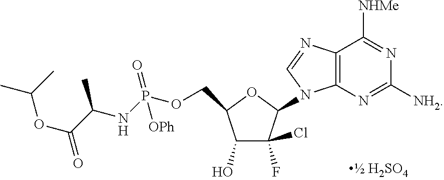 [SMILES: CNc1nc(N)nc2c1ncn2[C@@H]1O[C@H](COP(=O)(N[C@H](C)C(=O)OC(C)C)Oc2ccccc2)[C@@H](O)[C@]1(F)Cl]

65. The compound for use of embodiment 63 wherein the compound is:

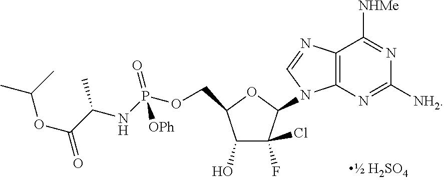 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@]1(F)Cl]

66. The compound for use of embodiment 63, wherein the compound is:

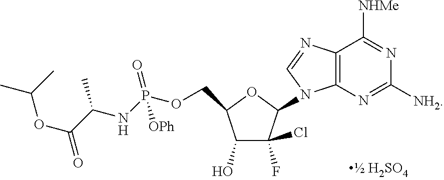 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@]1(F)Cl]

67. The compound for use of embodiment 64 wherein the compound is:

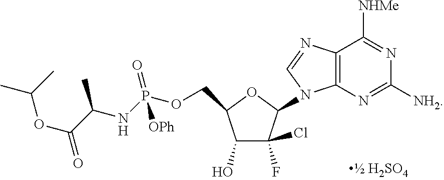 [SMILES: CNc1nc(N)nc2c1ncn2[C@@H]1O[C@H](CO[P@@](=O)(N[C@H](C)C(=O)OC(C)C)Oc2ccccc2)[C@@H](O)[C@]1(F)Cl]

68. The compound for use of embodiment 64, wherein the compound is:

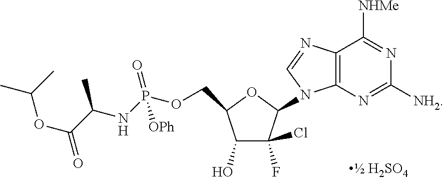 [SMILES: CNc1nc(N)nc2c1ncn2[C@@H]1O[C@H](CO[P@](=O)(N[C@H](C)C(=O)OC(C)C)Oc2ccccc2)[C@@H](O)[C@]1(F)Cl]

69. The compound for use of any of embodiments 47-68, wherein the virus is selected from dengue virus, West Nile fever, yellow fever virus, and Zika virus.

70. The compound for use of embodiment 69, wherein the virus is dengue virus.

71. The compound for use of embodiment 69, wherein the virus is yellow fever virus.

72. The compound for use of embodiment 69, wherein the virus is West Nile virus.

73. The compound for use of embodiment 69, wherein the virus is Zika virus.

74. The compound for use of any one of embodiments 47-73, wherein the compound is in a dosage form suitable for oral administration.

75. The compound for use of embodiment 74, wherein the oral dosage form is a solid oral dosage form.

76. The compound for use of embodiment 75, wherein the oral dosage form is a tablet.

77. The compound for use of embodiment 75, wherein the oral dosage form is a capsule.

78. The compound for use of any one of embodiments 47-77, wherein from about 500 mg to about 850 mg of the compound is administered.

79. The compound for use of any one of embodiments 47-77, wherein from about 500 mg to about 650 mg of the compound is administered.

80. The compound for use of any one of embodiments 47-77, wherein from about 600 mg to about 750 mg of the compound is administered.

81. The compound for use of any one of embodiments 47-77, wherein from about 650 mg to about 850 mg of the compound is administered.

82. The compound for use of any one of embodiments 47-77, wherein at least about 550 mg of the compound is administered.

83. The compound for use of any one of embodiments 47-77, wherein at least about 575 mg of the compound is administered.

84. The compound for use of any one of embodiments 47-77, wherein at least about 600 mg of the hemisulfate salt of the compound is administered.

85. The compound for use of any one of embodiments 47-77, wherein at least about 625 mg of the hemisulfate salt of the compound is administered.

86. The compound for use of any one of embodiments 47-77, wherein at least about 700 mg of the compound is administered.

87. The compound for use of any one of embodiments 47-77, wherein at least about 775 mg of the hemisulfate salt of the compound is administered.

88. The compound for use of any one of embodiments 47-87, wherein the compound is administered once per day.

89. The compound for use of any one of embodiments 47-87, wherein the compound is administered twice per day.

90. The compound for use of any one of embodiments 47-87, wherein the compound is

91. Use of a compound of Formula I:

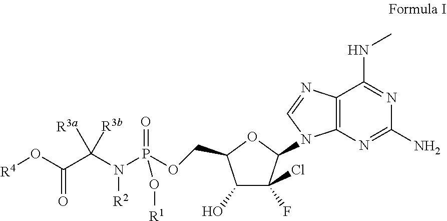

in the manufacture of a medicament for the treatment of an infection of a Flavivirus in a human host;
wherein:
R¹ is hydrogen, C₁₋₆alkyl, C₃₋₇cycloalkyl, aryl, —(C₁-C₄alkyl)aryl, heteroaryl, or heteroalkyl;
R² is hydrogen or C₁₋₆alkyl;
R^{3a} and R^{3b} are independently selected from hydrogen, C₁₋₆alkyl, and C₃₋₇cycloalkyl; and
R⁴ is hydrogen, C₁₋₆alkyl, C₁₋₆haloalkyl, C₃₋₇cycloalkyl, —(C₁-C₄alkyl)aryl, aryl, heteroaryl, or heteroalkyl;
or a pharmaceutically acceptable salt thereof.

92. The use of embodiment 91, wherein R¹ is hydrogen.

93. The use of embodiment 91, wherein R¹ is phenyl.

94. The use of any one of embodiments 91-93, wherein R² is hydrogen.

95. The use of any one of embodiments 91-94, wherein R^{3a} and R^{3b} are hydrogen and C₁₋₆alkyl.

96. The use of any one of embodiments 91-95 wherein R⁴ is C₁₋₆alkyl.

97. The use of embodiment 91,
wherein
R¹ is aryl;
R² is hydrogen;
R^{3a} is methyl; and
R⁴ is C₁₋₆alkyl.

98. The use of embodiment 91 wherein the compound is of formula:

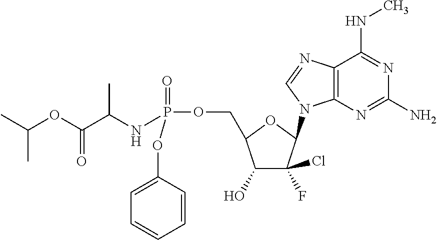 [SMILES: CNc1nc(N)nc2c1ncn2[C@@H]1OC(COP(=O)(NC(C)C(=O)OC(C)C)Oc2ccccc2)[C@@H](O)[C@]1(F)Cl]

or a pharmaceutically acceptable salt thereof.

99. The use of embodiment 98 wherein the compound is selected from:

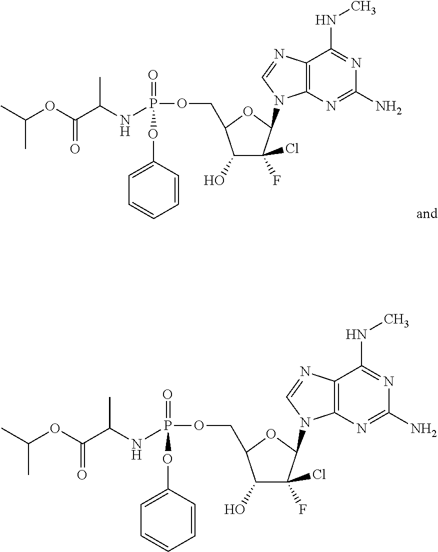 [SMILES: CNc1nc(N)nc2c1ncn2[C@@H]1OC(CO[P@](=O)(NC(C)C(=O)OC(C)C)Oc2ccccc2)[C@@H](O)[C@]1(F)Cl ; CNc1nc(N)nc2c1ncn2[C@@H]1OC(CO[P@@](=O)(NC(C)C(=O)OC(C)C)Oc2ccccc2)[C@@H](O)[C@]1(F)Cl]

or a pharmaceutically acceptable salt thereof.

100. The use of embodiment 98 wherein the compound is:

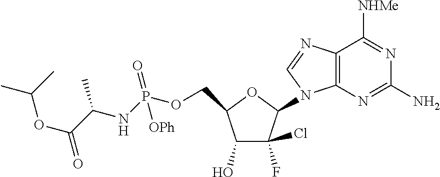 [SMILES: CNc1nc(N)nc2c1ncn2[C@@H]1O[C@H](COP(=O)(N[C@@H](C)C(=O)OC(C)C)Oc2ccccc2)[C@@H](O)[C@]1(F)Cl]

or a pharmaceutically acceptable salt thereof.

101. The use of embodiment 98 wherein the compound is:

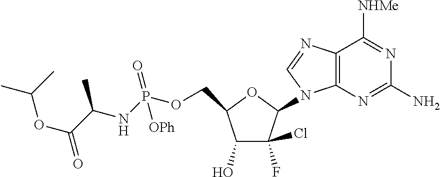 [SMILES: CNc1nc(N)nc2c1ncn2[C@@H]1O[C@H](COP(=O)(N[C@H](C)C(=O)OC(C)C)Oc2ccccc2)[C@@H](O)[C@]1(F)Cl]

or a pharmaceutically acceptable salt thereof.

102. The use of embodiment 100 wherein the compound is:

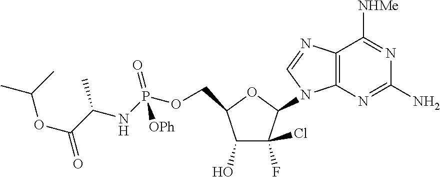 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@]1(F)Cl]

or a pharmaceutically acceptable salt thereof.

103. The use of embodiment 100, wherein the compound is:

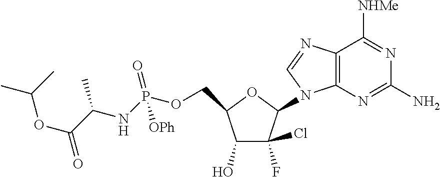 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@]1(F)Cl]

or a pharmaceutically acceptable salt thereof.

104. The use of embodiment 101 wherein the compound is:

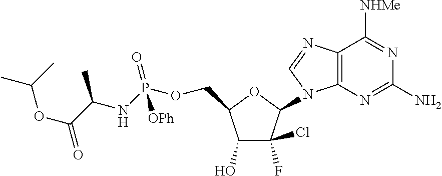 [SMILES: CNc1nc(N)nc2c1ncn2[C@@H]1O[C@H](CO[P@@](=O)(N[C@H](C)C(=O)OC(C)C)Oc2ccccc2)[C@@H](O)[C@]1(F)Cl]

or a pharmaceutically acceptable salt thereof.

105. The use of embodiment 101, wherein the compound is:

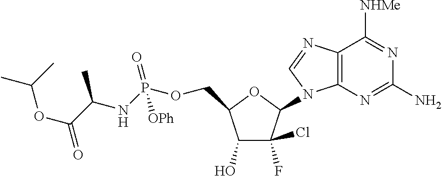 [SMILES: CNc1nc(N)nc2c1ncn2[C@@H]1O[C@H](CO[P@](=O)(N[C@H](C)C(=O)OC(C)C)Oc2ccccc2)[C@@H](O)[C@]1(F)Cl]

or a pharmaceutically acceptable salt thereof.

106. The use of any one of embodiments 91-105, wherein the pharmaceutically acceptable salt is the hemisulfate.

107. The use of embodiment 100 wherein the compound is:

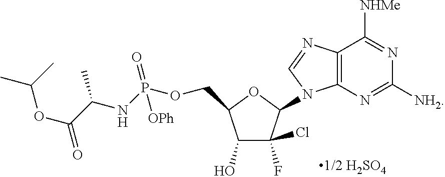 [SMILES: CNc1nc(N)nc2c1ncn2[C@@H]1O[C@H](COP(=O)(N[C@@H](C)C(=O)OC(C)C)Oc2ccccc2)[C@@H](O)[C@]1(F)Cl]

108. The use of embodiment 101 wherein the compound is:

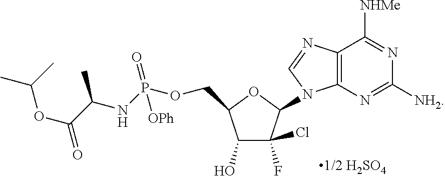 [SMILES: CNc1nc(N)nc2c1ncn2[C@@H]1O[C@H](COP(=O)(N[C@H](C)C(=O)OC(C)C)Oc2ccccc2)[C@@H](O)[C@]1(F)Cl]

109. The use of embodiment 107 wherein the compound is:

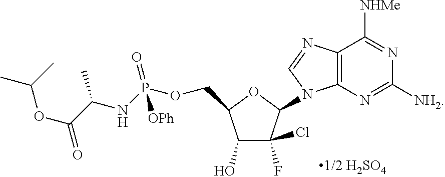 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@]1(F)Cl]

The use of embodiment 107, wherein the compound is: 110.

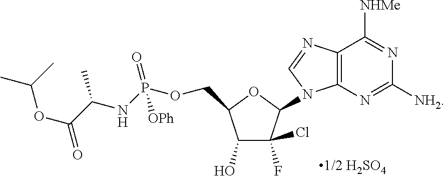 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@]1(F)Cl]

111. The use of embodiment 108 wherein the compound is:

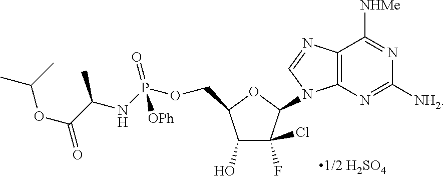 [SMILES: CNc1nc(N)nc2c1ncn2[C@@H]1O[C@H](CO[P@@](=O)(N[C@H](C)C(=O)OC(C)C)Oc2ccccc2)[C@@H](O)[C@]1(F)Cl]

112. The use of embodiment 108, wherein the compound is:

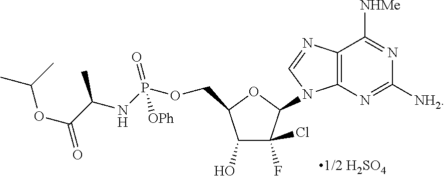 [SMILES: CNc1nc(N)nc2c1ncn2[C@@H]1O[C@H](CO[P@](=O)(N[C@H](C)C(=O)OC(C)C)Oc2ccccc2)[C@@H](O)[C@]1(F)Cl]

113. The use of any of embodiments 91-112, wherein the virus is selected from dengue virus, West Nile virus, yellow fever virus, and Zika virus.

114. The use of embodiment 113, wherein the virus is dengue virus.

115. The use of embodiment 113, wherein the virus is yellow fever virus.

116. The use of embodiment 113, wherein the virus is West Nile virus.

117. The use of embodiment 113, wherein the virus is Zika virus.

118. The use of any one of embodiments 91-117, wherein the compound is in a dosage form suitable for oral administration.

119. The use of embodiment 118, wherein the oral dosage form is a solid oral dosage form.

120. The use of embodiment 119, wherein the oral dosage form is a tablet.

121. The use of embodiment 119, wherein the oral dosage form is a capsule.

122. The use of any one of embodiments 91-121, wherein from about 500 mg to about 850 mg of the compound is administered.

123. The use of any one of embodiments 91-121, wherein from about 500 mg to about 650 mg of the compound is administered.

124. The use of any one of embodiments 91-121, wherein from about 600 mg to about 750 mg of the compound is administered.

125. The use of any one of embodiments 91-121, wherein from about 650 mg to about 850 mg of the compound is administered.

126. The use of any one of embodiments 91-121, wherein at least about 550 mg of the compound is administered.

127. The use of any one of embodiments 91-121, wherein at least about 575 mg of the compound is administered.

128. The use of any one of embodiments 91-121, wherein at least about 600 mg of the hemisulfate salt of the compound is administered.

129. The use of any one of embodiments 91-121, wherein at least about 625 mg of the hemisulfate salt of the compound is administered.

130. The use of any one of embodiments 91-121, wherein at least about 700 mg of the compound is administered.

131. The use of any one of embodiments 91-121, wherein at least about 775 mg of the hemisulfate salt of the compound is administered.

132. The use of any one of embodiments 91-131, wherein the compound is administered once per day.

133. The use of any one of embodiments 91-131, wherein the compound is administered twice per day.

134. The use of any one of embodiments 91-131, wherein the compound is

135. Use of an effective amount of a compound of Formula II:

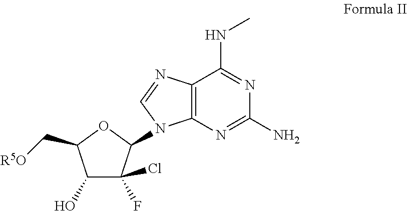

in the manufacture of a medicament for the treatment of an infection of a Flavivirus in a host in need thereof;
R⁵ is selected from

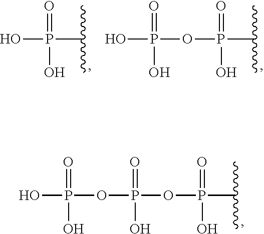

and R^{10A};
R^{10A} is a stabilized phosphate prodrug that metabolizes in vivo to a monophosphate, diphosphate, or triphosphate;
or a pharmaceutically acceptable salt thereof.

136. The use of embodiment 36 or 37, wherein the compound is of the formula

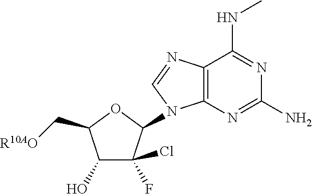

wherein
R¹⁰ª is a stabilized phosphate prodrug that metabolizes in vivo to a monophosphate, diphosphate, or triphosphate or pharmaceutically acceptable salt thereof.

137. The use of embodiment 135 or 136, wherein the virus is selected from dengue virus, West Nile virus, yellow fever virus, and Zika virus.

138. The use of embodiment 137, wherein the virus is dengue virus.

139. The use of embodiment 137, wherein the virus is yellow fever virus.

140. The use of embodiment 137, wherein the virus is Zika virus.

141. The use of embodiment 137, wherein the virus is West Nile virus.

142. The use of embodiment 137, wherein the host is a human.

143. A pharmaceutical composition for use to treat a human host in need thereof infected with a Flavivirus, comprising an effective amount of Formula I:

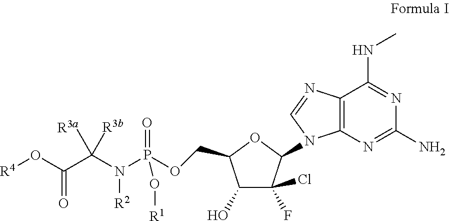

wherein:
R¹ is hydrogen, C₁₋₆alkyl, C₃₋₇cycloalkyl, aryl, —(C₁-C₄alkyl)aryl, heteroaryl, or heteroalkyl;
R² is hydrogen or C₁₋₆alkyl;
R^{3a} and R^{3b} are independently selected from hydrogen, C₁₋₆alkyl, and C₃₋₇cycloalkyl; and
R⁴ is hydrogen, C₁₋₆alkyl, C₁₋₆haloalkyl, C₃₋₇cycloalkyl, —(C₁-C₄alkyl)aryl, aryl, heteroaryl, or heteroalkyl;
or a pharmaceutically acceptable salt thereof.

144. The pharmaceutical composition for use of embodiment 143, wherein R¹ is hydrogen.

145. The pharmaceutical composition for use of embodiment 143, wherein R¹ is phenyl.

146. The pharmaceutical composition for use of any one of embodiments 143-145, 146, wherein R² is hydrogen.

147. The pharmaceutical composition for use of any one of embodiments 143-146, wherein R^{3a} and R^{3b} are hydrogen and C₁₋₆alkyl.

148. The pharmaceutical composition for use of any one of embodiments 143-147 wherein R⁴ is C₁₋₆alkyl.

149. The pharmaceutical composition for use of embodiment 143,
wherein
R¹ is aryl;
R² is hydrogen;
R^{3a} is methyl; and
R⁴ is C₁₋₆alkyl.

150. The pharmaceutical composition for use of embodiment 143 wherein the compound is of formula:

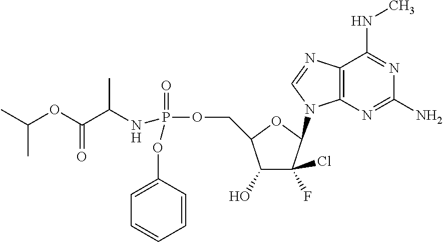 [SMILES: CNc1nc(N)nc2c1ncn2[C@@H]1OC(COP(=O)(NC(C)C(=O)OC(C)C)Oc2ccccc2)[C@@H](O)[C@]1(F)Cl]

or a pharmaceutically acceptable salt thereof.

151. The pharmaceutical composition for use of embodiment 150 wherein the compound is selected from:

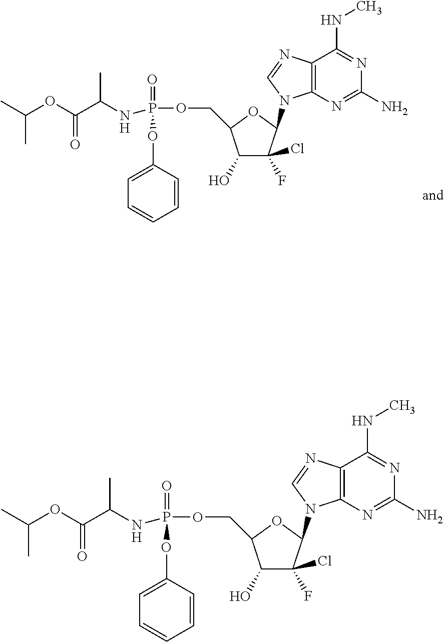 [SMILES: CNc1nc(N)nc2c1ncn2[C@@H]1OC(CO[P@](=O)(NC(C)C(=O)OC(C)C)Oc2ccccc2)[C@@H](O)[C@]1(F)Cl ; CNc1nc(N)nc2c1ncn2[C@@H]1OC(CO[P@@](=O)(NC(C)C(=O)OC(C)C)Oc2ccccc2)[C@@H](O)[C@]1(F)Cl]

or a pharmaceutically acceptable salt thereof.

152. The pharmaceutical composition for use of embodiment 150 wherein the compound is:

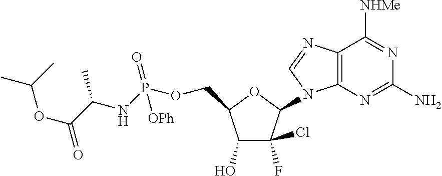 [SMILES: CNc1nc(N)nc2c1ncn2[C@@H]1O[C@H](COP(=O)(N[C@@H](C)C(=O)OC(C)C)Oc2ccccc2)[C@@H](O)[C@]1(F)Cl]

or a pharmaceutically acceptable salt thereof.

153. The pharmaceutical composition for use of embodiment 150 wherein the compound is:

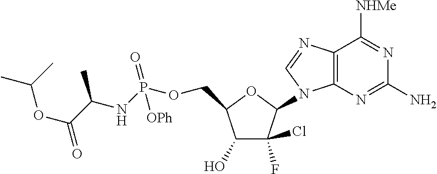 [SMILES: CNc1nc(N)nc2c1ncn2[C@@H]1O[C@H](COP(=O)(N[C@H](C)C(=O)OC(C)C)Oc2ccccc2)[C@@H](O)[C@]1(F)Cl]

or a pharmaceutically acceptable salt thereof.

154. The pharmaceutical composition for use of embodiment 152 wherein the compound is:

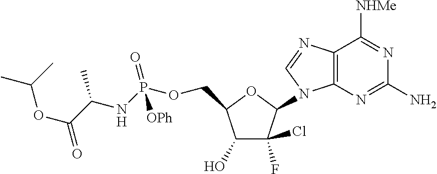 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@]1(F)Cl]

or a pharmaceutically acceptable salt thereof.

155. The pharmaceutical composition for use of embodiment 152, wherein the compound is:

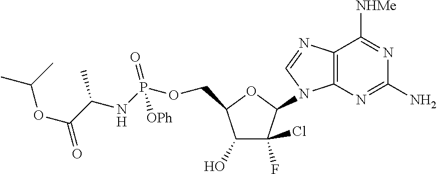 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@]1(F)Cl]

or a pharmaceutically acceptable salt thereof.

156. The pharmaceutical composition for use of embodiment 151 wherein the compound is:

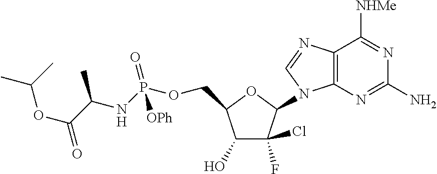 [SMILES: CNc1nc(N)nc2c1ncn2[C@@H]1O[C@H](CO[P@@](=O)(N[C@H](C)C(=O)OC(C)C)Oc2ccccc2)[C@@H](O)[C@]1(F)Cl]

or a pharmaceutically acceptable salt thereof.

157. The pharmaceutical composition for use of embodiment 151, wherein the compound is:

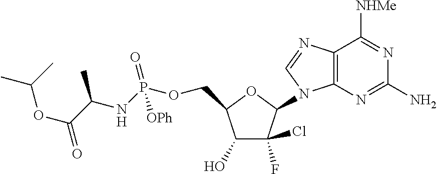 [SMILES: CNc1nc(N)nc2c1ncn2[C@@H]1O[C@H](CO[P@](=O)(N[C@H](C)C(=O)OC(C)C)Oc2ccccc2)[C@@H](O)[C@]1(F)Cl]

or a pharmaceutically acceptable salt thereof.

158. The pharmaceutical composition for use of embodiment 152 wherein the compound is:

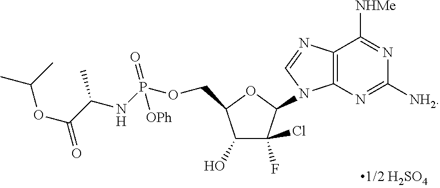 [SMILES: CNc1nc(N)nc2c1ncn2[C@@H]1O[C@H](COP(=O)(N[C@@H](C)C(=O)OC(C)C)Oc2ccccc2)[C@@H](O)[C@]1(F)Cl]

159. The pharmaceutical composition for use of embodiment 153 wherein the compound is:

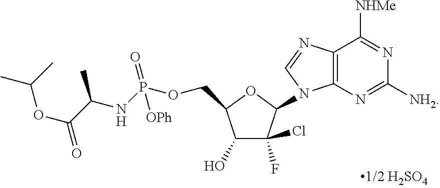 [SMILES: CNc1nc(N)nc2c1ncn2[C@@H]1O[C@H](COP(=O)(N[C@H](C)C(=O)OC(C)C)Oc2ccccc2)[C@@H](O)[C@]1(F)Cl]

160. The pharmaceutical composition for use of embodiment 158 wherein the compound is:

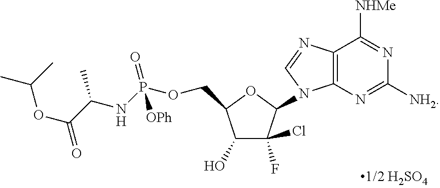 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@]1(F)Cl]

161. The pharmaceutical composition for use of embodiment 158, wherein the compound is:

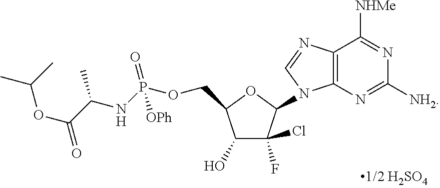 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@]1(F)Cl]

162. The pharmaceutical composition for use of embodiment 159 wherein the compound is:

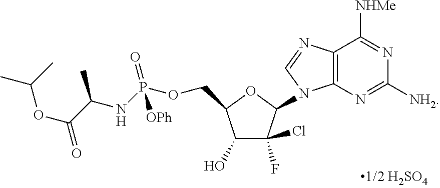 [SMILES: CNc1nc(N)nc2c1ncn2[C@@H]1O[C@H](CO[P@@](=O)(N[C@H](C)C(=O)OC(C)C)Oc2ccccc2)[C@@H](O)[C@]1(F)Cl]

163. The pharmaceutical composition for use of embodiment 159, wherein the compound is:

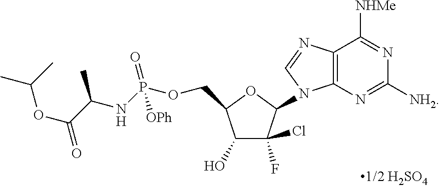 [SMILES: CNc1nc(N)nc2c1ncn2[C@@H]1O[C@H](CO[P@](=O)(N[C@H](C)C(=O)OC(C)C)Oc2ccccc2)[C@@H](O)[C@]1(F)Cl]

164. The pharmaceutical composition for use of any of embodiments 150-163, wherein the virus is selected from dengue virus, West Nile fever, yellow fever virus, and Zika virus.

165. The pharmaceutical composition for use of embodiment 164, wherein the virus is dengue virus.

166. The pharmaceutical composition for use of embodiment 164, wherein the virus is yellow fever virus.

167. The pharmaceutical composition for use of embodiment 164, wherein the virus is West Nile virus.

168. The pharmaceutical composition for use of embodiment 164, wherein the virus is Zika virus.

169. The pharmaceutical composition for use of any one of embodiments 150-168, wherein the compound is in a dosage form suitable for oral administration.

170. The pharmaceutical composition for use of embodiment 169, wherein the oral dosage form is a solid oral dosage form.

171. The pharmaceutical composition for use of embodiment 169, wherein the oral dosage form is a tablet.

172. The pharmaceutical composition for use of embodiment 169, wherein the oral dosage form is a capsule.

173. The pharmaceutical composition for use of any one of embodiments 150-172, wherein from about 500 mg to about 850 mg of the compound is administered.

174. The pharmaceutical composition for use of any one of embodiments 150-172, wherein from about 500 mg to about 650 mg of the compound is administered.

175. The pharmaceutical composition for use of any one of embodiments 150-172, wherein from about 600 mg to about 750 mg of the compound is administered.

176. The pharmaceutical composition for use of any one of embodiments 150-172, wherein from about 650 mg to about 850 mg of the compound is administered.

177. The pharmaceutical composition for use of any one of embodiments 150-172, wherein at least about 550 mg of the compound is administered.

178. The pharmaceutical composition for use of any one of embodiments 150-172, wherein at least about 575 mg of the compound is administered.

179. The pharmaceutical composition for use of any one of embodiments 150-172, wherein at least about 600 mg of the hemisulfate salt of the compound is administered.

180. The pharmaceutical composition for use of any one of embodiments 150-172, wherein at least about 625 mg of the hemisulfate salt of the compound is administered.

181. The pharmaceutical composition for use of any one of embodiments 150-172, wherein at least about 700 mg of the compound is administered.

182. The pharmaceutical composition for use of any one of embodiments 150-172, wherein at least about 775 mg of the hemisulfate salt of the compound is administered.

183. The pharmaceutical composition for use of any one of embodiments 150-182, wherein the compound is administered once per day.

184. The pharmaceutical composition for use of any one of embodiments 150-182, wherein the compound is administered twice per day.

185. The pharmaceutical composition for use of any one of embodiments 150-182, wherein the compound is administered four times per day.

### Stabilized Phosphate Prodrugs

Stabilized phosphate prodrugs are moieties that can deliver a mono, di, or triphosphate in vivo. For example, McGuigan has disclosed phosphoramidates in U.S. Pat. Nos. 8,933,053; 8,759,318; 8,658,616; 8,263,575; 8,119,779; 7,951,787 and 7,115,590. Alios has disclosed thiophosphoramidates in U.S. Pat. Nos. 8,895,723 and 8,871,737 incorporated by reference herein. Alios has also disclosed cyclic nucleotides in U.S. Pat. No. 8,772,474 incorporated by reference herein. Idenix has disclosed cyclic phosphoramidates and phosphoramidate/SATE derivatives in WO 2013/177219 incorporated by reference herein. Idenix has also disclosed substituted carbonyloxymethylphosphoramidate compounds in WO 2013/039920 incorporated by reference herein. Hostetler has disclosed lipid phosphate prodrugs, see, for example, U.S. Pat. No. 7,517,858, incorporated by reference herein. Hostetler has also disclosed lipid conjugates of phosphonate prodrugs, see, for example, U.S. Pat. Nos. 8,889,658; 8,846,643; 8,710,030; 8,309,565; 8,008,308; and 7,790,703. Emory University has disclosed nucleotide sphingoid and lipid derivatives in WO 2014/124430 incorporated by reference herein. RFS Pharma has disclosed purine nucleoside monophosphate prodrugs in WO 2010/091386. Cocrystal Pharma Inc. has also disclosed purine nucleoside monophosphate prodrugs in U.S. Pat. No. 9,173,893 incorporated by reference herein. HepDirect™ technology is disclosed in the article “Design, Synthesis, and Characterization of a Series of Cytochrome P(450) 3A-Activated Prodrugs (HepDirect Prodrugs) Useful for Targeting Phosph(on)ate-Based Drugs to the Liver,” (J. Am. Chem. Soc. 126, 5154-5163 (2004). Additional phosphate prodrugs include, but are not limited to phosphate esters, 3′,5′-cyclic phosphates including CycloSAL, SATE derivatives (S-acyl-2-thioesters) and DTE (dithiodiethyl) prodrugs. For literature reviews that disclose non-limiting examples see: A. Ray and K. Hostetler, “Application of kinase bypass strategies to nucleoside antivirals,” Antiviral Research (2011) 277-291; M. Sofia, “Nucleotide prodrugs for HCV therapy,” Antiviral Chemistry and Chemotherapy 2011; 22-23-49; and S. Peyrottes et al., “SATE Pronucleotide Approaches: An Overview,” Mini Reviews in Medicinal Chemistry 2004, 4, 395. In certain embodiments, a 5′-prodrug described in any of these patent filings or literature can be used in the R⁵ position of the presented compounds.

In certain embodiments, the stabilized phosphate prodrugs, include, but are not limited to those described in U.S. Pat. Nos. 9,173,893 and 8,609,627, incorporated by reference herein, including for processes of preparation. For example, 5′-prodrugs can be represented by the group:

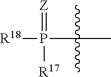

wherein
Z is O or S;
R¹⁷ and R¹⁸, when administered in vivo, are capable of providing the nucleoside monophosphate, diphosphate, or triphosphate. Representative R¹⁷ and R¹⁸ are independently selected from:
(a) OR¹⁹ where R¹⁹ is selected from H, Li, Na, K, phenyl and pyridinyl and wherein phenyl and pyridinyl are optionally substituted with one to three substituents independently selected from the group consisting of (CH₂)₀₋₆CO₂R²⁰ and (CH₂)₀₋₆CON(R²⁰)₂;
R²⁰ is independently H, C₁₋₂₀ alkyl, the carbon chain derived from a fatty alcohol (such as oleyl alcohol, octacosanol, triacontanol, linoleyl alcohol, and etc.) or C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;

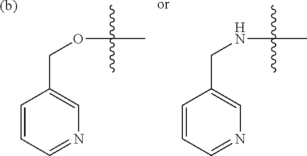 [SMILES: "(b)" ; "or" ; *OCc1cccnc1 ; *NCc1cccnc1]

(c) the ester of a D-amino acid or L-amino acid:

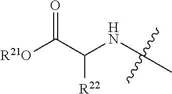

wherein
R²¹ is restricted to those sidechains occurring in natural L-amino acids, and
R²² is H, C₁₋₂₀ alkyl, the carbon chain derived from a fatty alcohol (such as oleyl alcohol, octacosanol, triacontanol, linoleyl alcohol, and etc.) or C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
   (d) R¹⁷ and R¹⁸ can come together to form a ring:

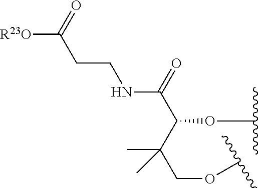

wherein
R²³ is H, C₁₋₂₀ alkyl, C₁₋₂₀ alkenyl, the carbon chain derived from a fatty alcohol (such as oleyl alcohol, octacosanol, triacontanol, linoleyl alcohol, etc.) or C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
   (e) R¹⁷ and R¹⁸ can come together to form a ring selected from

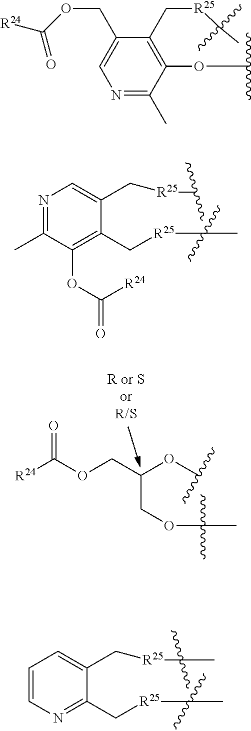

wherein
R²⁴ is selected from H, C₁₋₂₀ alkyl, C₁₋₂₀ alkenyl, the carbon chain derived from a fatty acid (such as oleic acid, linoleic acid, and the like), and C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl; and
R²⁵ is O or NH.

The present invention also includes administration of an effective amount of a compound of Formula II wherein R⁵ is a monophosphate, a diphosphate, a triphosphate, or R^{10A}, wherein R^{10A} is a stabilized phosphate prodrug that metabolizes in vivo to a monophosphate, diphosphate, or triphosphate to treat infections of a Flavivirus in a host in need thereof, typically a human, as described herein:

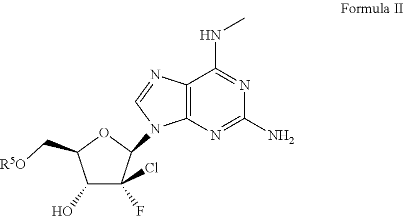

wherein:
R⁵ is selected from

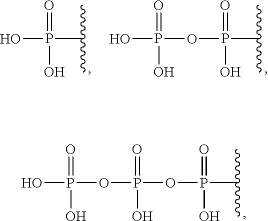

### and R^{10A};

R^{10A} is a stabilized phosphate prodrug that metabolizes in vivo to a monophosphate, diphosphate, or triphosphate;
or pharmaceutically acceptable salt thereof, and
all other variables are as previously defined herein.

In some embodiments, R⁵ is selected from

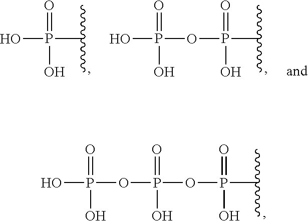

In some embodiments, R⁵ is

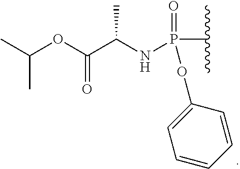 [SMILES: *P(=O)(N[C@@H](C)C(=O)OC(C)C)Oc1ccccc1]

In some embodiments, R⁵ is

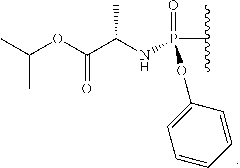 [SMILES: *[P@@](=O)(N[C@@H](C)C(=O)OC(C)C)Oc1ccccc1]

In some embodiments, R⁵ is

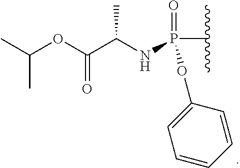 [SMILES: *[P@](=O)(N[C@@H](C)C(=O)OC(C)C)Oc1ccccc1]

In one embodiment of Formula II, R⁵ is

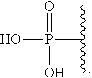 [SMILES: *P(=O)(O)O]

In one embodiment of Formula II, R⁵ is

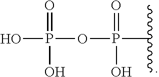 [SMILES: *P(=O)(O)OP(=O)(O)O]

In one embodiment of Formula II, R⁵ is

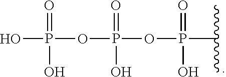 [SMILES: *P(=O)(O)OP(=O)(O)OP(=O)(O)O]

In one embodiment of Formula II, R⁵ is R^{10A}.

### EMBODIMENTS

(i). In one embodiment of Formula I, R¹ is aryl, R² is hydrogen, R^{3a} is hydrogen, R^{3b} is C₁-C₄alkyl, and R⁴ is C₁-C₆alkyl.
(ii). In one embodiment of Formula I, R¹ is aryl, R² is hydrogen, R^{3a} is hydrogen, R^{3b} is methyl, and R⁴ is C₁-C₆alkyl.
(iii). In one embodiment of Formula I, R¹ is phenyl, R² is hydrogen, R^{3a} is hydrogen, R^{3b} is C₁-C₄alkyl, and R⁴ is C₁-C₆alkyl.
(iv). In one embodiment of Formula I, R¹ is phenyl, R² is hydrogen, R^{3a} is hydrogen, R^{3b} is methyl, and R⁴ is C₁-C₆alkyl.
(v). In one embodiment of Formula I, R¹ is aryl, R² is methyl, R^{3a} is hydrogen, R^{3b} is C₁-C₄alkyl, and R⁴ is C₁-C₆alkyl.
(vi). In one embodiment of Formula I, R¹ is aryl, R² is methyl, R^{3a} is hydrogen, R^{3b} is methyl, and R⁴ is C₁-C₆alkyl.
(vii). In one embodiment of Formula I, R¹ is phenyl, R² is methyl, R^{3a} is hydrogen, R^{3b} is C₁-C₄alkyl, and R⁴ is C₁-C₆alkyl.
(viii). In one embodiment of Formula I, R¹ is phenyl, R² is methyl, R^{3a} is hydrogen, R^{3b} is methyl, and R⁴ is C₁-C₆alkyl.
(ix). In one embodiment of Formula I, R¹ is aryl, R² is hydrogen, R^{3a} is hydrogen, R^{3b} is C₁-C₄alkyl, and R⁴ is isopropyl.
(x). In one embodiment of Formula I, R¹ is aryl, R² is hydrogen, R^{3a} is hydrogen, R^{3b} is methyl, and R⁴ is isopropyl.
(xi). In one embodiment of Formula I, R¹ is phenyl, R² is hydrogen, R^{3a} is hydrogen, R^{3b} is C₁-C₄alkyl, and R⁴ is isopropyl.
(xii). In one embodiment of Formula I, R¹ is phenyl, R² is hydrogen, R^{3a} is hydrogen, R^{3b} is methyl, and R⁴ is isopropyl.
(xiii). In one embodiment of Formula I, R¹ is aryl, R² is methyl, R^{3a} is hydrogen, R^{3b} is C₁-C₄alkyl, and R⁴ is isopropyl.
(xiv). In one embodiment of Formula I, R¹ is aryl, R² is methyl, R^{3a} is hydrogen, R^{3b} is methyl, and R⁴ is isopropyl.
(xv). In one embodiment of Formula I, R¹ is phenyl, R² is methyl, R^{3a} is hydrogen, R^{3b} is C₁-C₄alkyl, and R⁴ is isopropyl.
(xvi). In one embodiment of Formula I, R¹ is phenyl, R² is methyl, R^{3a} is hydrogen, R^{3b} is methyl, and R⁴ is isopropyl.

In certain embodiments of (i) through (xvi), an L-nucleoside is used in Formula I.

In certain embodiments, treatment of an infection of a Flavivirus in a host, including a human, in need thereof comprises the administration of an effective amount of a compound of Formula I or a pharmaceutically acceptable salt thereof. In certain embodiments, the pharmaceutically acceptable salt of a compound of Formula I is the hemisulfate salt. Additional non-limiting examples of a compound of Formula I include

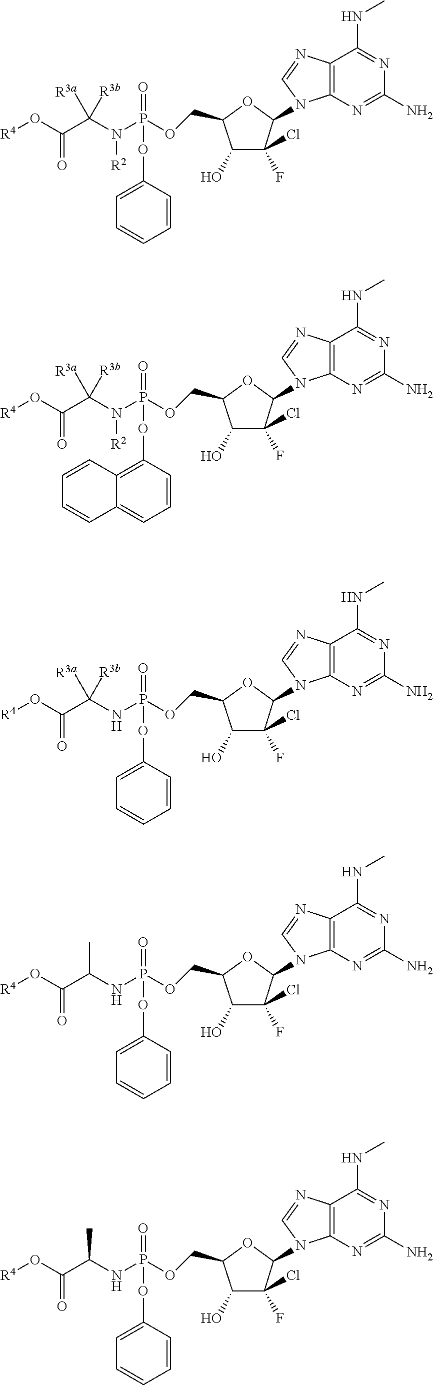

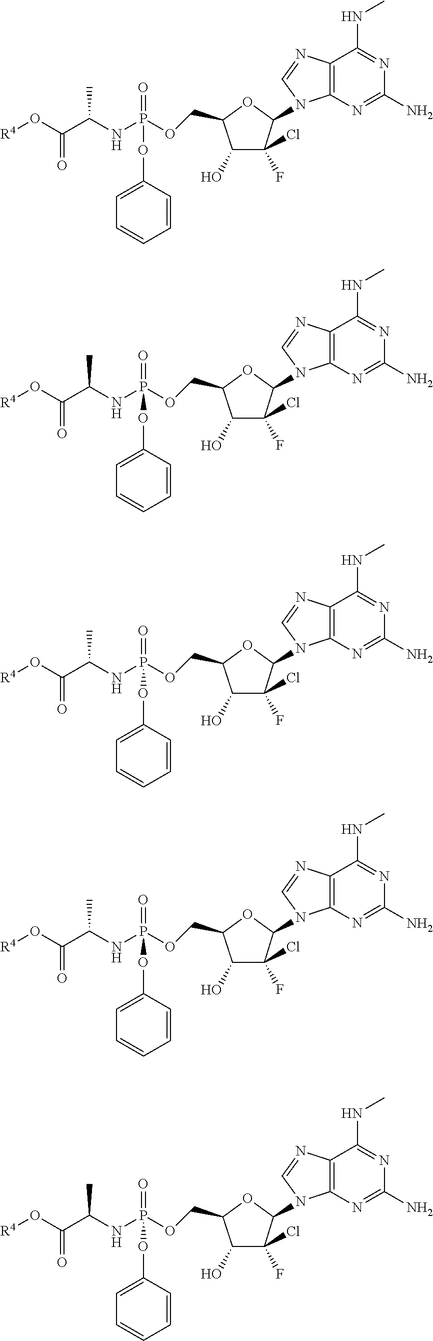

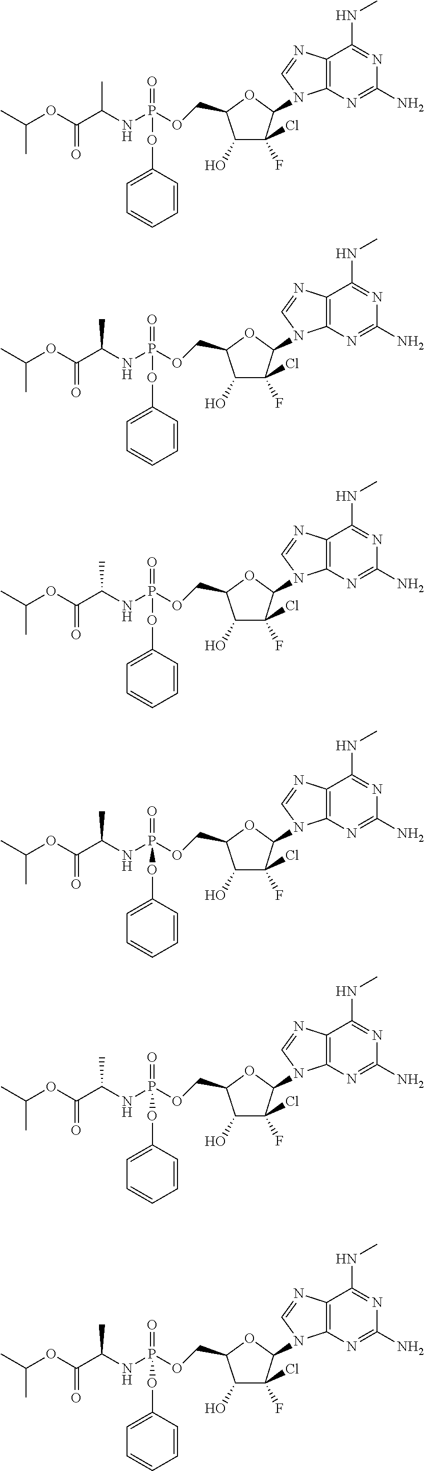

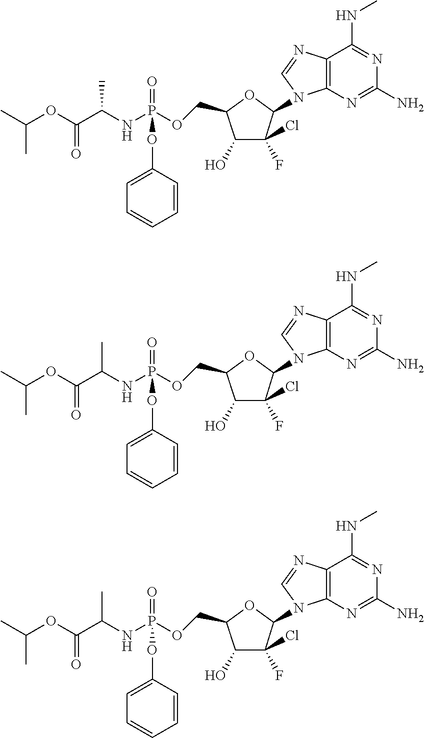 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@]1(F)Cl ; CNc1nc(N)nc2c1ncn2[C@@H]1O[C@H](CO[P@@](=O)(NC(C)C(=O)OC(C)C)Oc2ccccc2)[C@@H](O)[C@]1(F)Cl ; CNc1nc(N)nc2c1ncn2[C@@H]1O[C@H](CO[P@](=O)(NC(C)C(=O)OC(C)C)Oc2ccccc2)[C@@H](O)[C@]1(F)Cl]

In certain embodiments, a treatment of an infection of a Flavivirus in a host, including a human, in need thereof comprises the administration of an effective amount of a compound of Formula I or a pharmaceutically acceptable salt thereof. In certain embodiments, the pharmaceutically acceptable salt of a compound of Formula I is the hemisulfate salt. Additional non-limiting examples of a compound of Formula I include

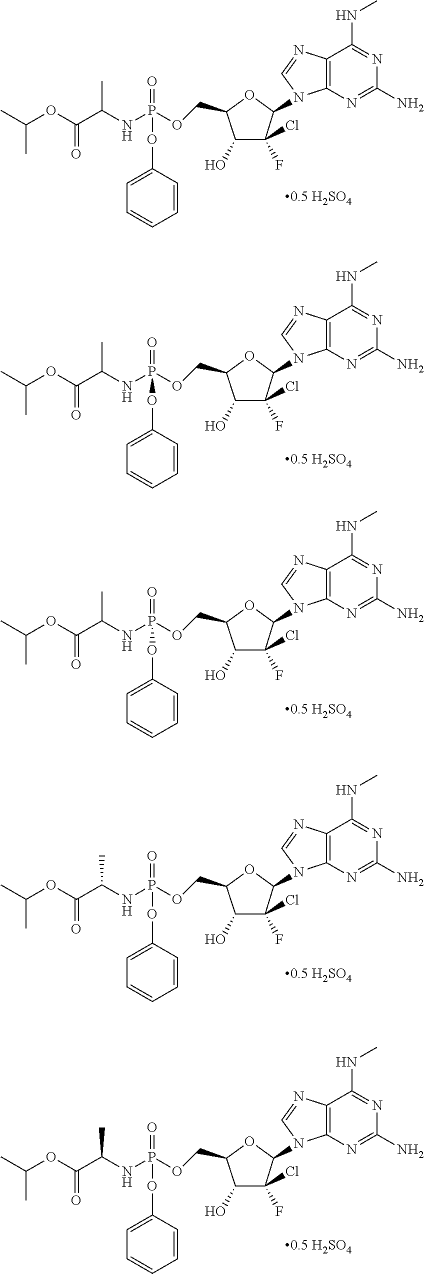 [SMILES: CNc1nc(N)nc2c1ncn2[C@@H]1O[C@H](COP(=O)(NC(C)C(=O)OC(C)C)Oc2ccccc2)[C@@H](O)[C@]1(F)Cl ; CNc1nc(N)nc2c1ncn2[C@@H]1O[C@H](CO[P@@](=O)(NC(C)C(=O)OC(C)C)Oc2ccccc2)[C@@H](O)[C@]1(F)Cl ; CNc1nc(N)nc2c1ncn2[C@@H]1O[C@H](CO[P@](=O)(NC(C)C(=O)OC(C)C)Oc2ccccc2)[C@@H](O)[C@]1(F)Cl ; CNc1nc(N)nc2c1ncn2[C@@H]1O[C@H](COP(=O)(N[C@@H](C)C(=O)OC(C)C)Oc2ccccc2)[C@@H](O)[C@]1(F)Cl ; CNc1nc(N)nc2c1ncn2[C@@H]1O[C@H](COP(=O)(N[C@H](C)C(=O)OC(C)C)Oc2ccccc2)[C@@H](O)[C@]1(F)Cl]

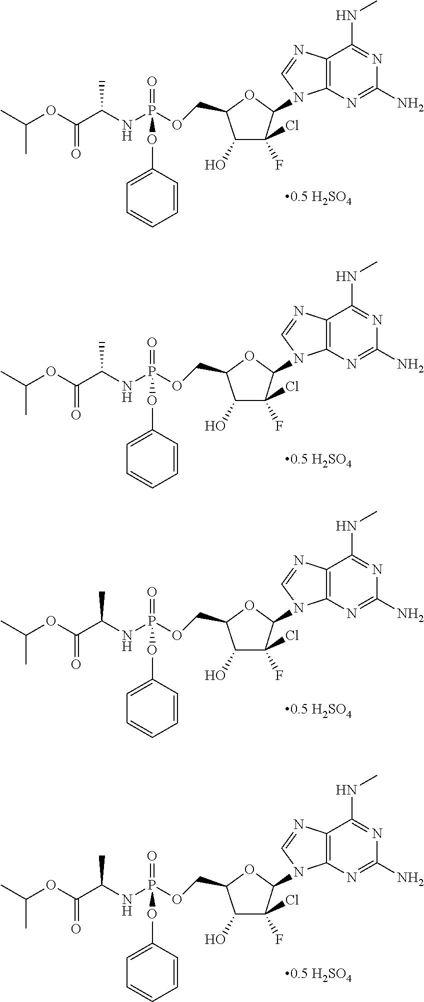 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@]1(F)Cl ; CNc1nc(N)nc2c1ncn2[C@@H]1O[C@H](CO[P@](=O)(N[C@@H](C)C(=O)OC(C)C)Oc2ccccc2)[C@@H](O)[C@]1(F)Cl ; CNc1nc(N)nc2c1ncn2[C@@H]1O[C@H](CO[P@](=O)(N[C@H](C)C(=O)OC(C)C)Oc2ccccc2)[C@@H](O)[C@]1(F)Cl ; CNc1nc(N)nc2c1ncn2[C@@H]1O[C@H](CO[P@@](=O)(N[C@H](C)C(=O)OC(C)C)Oc2ccccc2)[C@@H](O)[C@]1(F)Cl]

### II. Definitions

The following terms are used to describe the present invention. In instances where a term is not specifically defined herein, that term is given an art-recognized meaning by those of ordinary skill applying that term in context to its use in describing the present invention.

The term “alkyl” shall mean within its context, a linear, or branch-chained fully saturated hydrocarbon radical or alkyl group which can be optionally substituted (for example, with halogen, including F). For example, an alkyl group can have 1, 2, 3, 4, 5, 6, 7 or 8 carbon atoms (i.e., C₁-C₈ alkyl), 1, 2, 3, 4, 5 or 6 carbon atoms (i.e., C₁-C₆ alkyl) or 1 to 4 carbon atoms (i.e., C₁-C₄ alkyl). Examples of suitable alkyl groups include, but are not limited to, methyl, ethyl, n-propyl, iso-propyl, n-butyl, isobutyl, sec-butyl, tert-butyl, pentyl, isopentyl, tert-pentyl, neopentyl, hexyl, 2-methylpentyl, 3-methylpentyl, 2,2-dimethylbutyl and 2,3-dimethylbutyl.

The term “alkenyl” refers to a non-aromatic hydrocarbon group which contains at least one double bond between adjacent carbon atoms and a similar structure to an alkyl group as otherwise described herein. For example, an alkenyl group can have 2 to 8 carbon atoms (i.e., C₂-C₈ alkenyl), or 2 to 4 carbon atoms (i.e., C₂-C₄ alkenyl). Examples of suitable alkenyl groups include, but are not limited to, ethenyl or vinyl (—CH═CH₂), allyl (—CH₂CH═CH₂), 1-butenyl (—C═CH—CH₂CH₃) and 2-butenyl (—CH₂CH═CHCH₂). The alkenyl group can be optionally substituted as described herein.

The term “alkynyl” refers to a non-aromatic hydrocarbon group containing at least one triple bond between adjacent carbon atoms and a similar structure to an alkyl group as otherwise described herein. For example, an alkynyl group can have 2 to 8 carbon atoms (i.e., C₂-C₈ alkyne,), or 2 to 4 carbon atoms (i.e., C₂-C₄ alkynyl). Examples of alkynyl groups include, but are not limited to, acetylenic or ethynyl and propargyl. The alkynyl group can be optionally substituted as described herein.

The term “acyl” refers to the moiety —C(O)R in which the carbonyl moiety is bonded to R, for example, —C(O)alkyl. R can be selected from alkoxy, alkyl, cycloalkyl, lower alkyl (i.e., C₁-C₄); alkoxyalkyl, including methoxymethyl; aralkyl—including benzyl, aryloxyalkyl—such as phenoxymethyl; aryl including phenyl optionally substituted with halogen, C₁ to C₄ alkyl or C₁ to C₄ alkoxy. In one embodiment, the term “acyl” refers to a mono, di or triphosphate.

The term “lower acyl” refers to an acyl group in which the carbonyl moiety is lower alkyl (i.e., C₁-C₄).

The term “alkoxy” refers to the group —OR′ where —OR′ is —O-alkyl, —O-alkenyl, —O— alkynyl, —O—(C₀-C₂)(cycloalkyl), —O—(C₀-C₂)(heterocyclo), —O—(C₀-C₂)(aryl), or —O—(C₀-C₂)(heteroaryl), each of which can be optionally substituted.

The term “amino” refers to the group —NH₂.

The term “amino acid” or “amino acid residue” refers to a D- or L-natural or non-naturally occurring amino acid. Representative amino acids include, but are not limited to, alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan, or tyrosine, among others.

The term “aryl” or “aromatic”, in context, refers to a substituted (as otherwise described herein) or unsubstituted monovalent aromatic radical having a single ring (e.g., phenyl or benzyl) or condensed rings (e.g., naphthyl, anthracenyl, phenanthrenyl, etc.) and can be bound to the compound according to the present invention at any available stable position on the ring(s) or as otherwise indicated in the chemical structure presented. The aryl group can be optionally substituted as described herein.

“Cycloalkyl”, “carbocycle”, or “carbocyclyl” refers to a saturated (i.e., cycloalkyl) or partially unsaturated (e.g., cycloakenyl, cycloalkadienyl, etc.) ring having 3 to 7 carbon atoms as a monocycle. Monocyclic carbocycles have 3 to 7 ring atoms, still more typically 5 or 6 ring atoms. Non-limiting examples of cycloalkyl groups include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, and 1-cyclo-hex-3-enyl.

The term “cyano” refers to the group —CN.

The term “halogen” or “halo” refers to chloro, bromo, fluoro or iodo.

A heteroaryl ring system is a saturated or unsaturated ring with one or more nitrogen, oxygen, or sulfur atoms in the ring (monocyclic) including but not limited to imidazole, furyl, pyrrole, furanyl, thiene, thiazole, pyridine, pyrimidine, purine, pyrazine, triazole, oxazole, or fused ring systems such as indole, quinoline, etc., among others, which may be optionally substituted as described above. Heteroaryl groups include nitrogen-containing heteroaryl groups such as pyrrole, pyridine, pyridone, pyridazine, pyrimidine, pyrazine, pyrazole, imidazole, triazole, triazine, tetrazole, indole, isoindole, indolizine, purine, indazole, quinoline, isoquinoline, quinolizine, phthalazine, naphthyridine, quinoxaline, quinazoline, cinnoline, pteridine, imidazopyridine, imidazotriazine, pyrazino-pyridazine, acridine, phenanthridine, carbazole, carbazoline, perimidine, phenanthroline, phenacene, oxadiazole, benzimidazole, pyrrolopyridine, pyrrolopyrimidine and pyridopyrimidine; sulfur-containing aromatic heterocycles such as thiophene and benzothiophene; oxygen-containing aromatic heterocycles such as furan, pyran, cyclopentapyran, benzofuran and isobenzofuran; and aromatic heterocycles comprising two or more hetero atoms selected from among nitrogen, sulfur and oxygen, such as thiazole, thiadiazole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole, among others, all of which may be optionally substituted.

The term “heterocycle” or “heterocyclo” refers to a cyclic group which contains at least one heteroatom, i.e., O, N, or S, and may be aromatic (heteroaryl) or non-aromatic. Exemplary non-aromatic heterocyclic groups for use in the present invention include, for example, pyrrolidinyl, piperidinyl, piperazinyl, N-methylpiperazinyl, imidazolinyl, pyrazolidinyl, imidazolidinyl, morpholinyl, tetrahydropyranyl, azetidinyl, oxetanyl, oxathiolanyl, pyridone, 2-pyrrolidone, ethylene urea, 1,3-dioxolane, 1,3-dioxane, 1,4-dioxane, phthalimide, and succinimide, among others, all of which may be optionally substituted.

The term “hydroxyl” refers to the group —OH.

The term “nitro” refers to the group —NO₂.

The term “pharmaceutically acceptable salt” or “prodrug” is used throughout the specification to describe the salt of any pharmaceutically acceptable form (such as an ester, phosphoramidate, thiophosphoramidate, phosphate ester, salt of an ester, or a related group) of a 2′-chloro-2′-fluoro-N²-amino-N⁶-methylamino purine nucleotide which, upon administration to a patient, provides the desired active compound. Examples of pharmaceutically acceptable salts are organic acid addition salts formed with acids, which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including sulfate, nitrate, bicarbonate, and carbonate salts. Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium, or lithium) or alkaline earth metal (for example calcium) salts of carboxylic acids can also be made.

“Pharmaceutically acceptable prodrug” refers to a compound that is metabolized, for example hydrolyzed or oxidized, in the host to form the compound of the present invention. Typical examples of prodrugs include a compound that has biologically labile protecting groups on a functional moiety of the active compound. Prodrugs include a compound that can be oxidized, reduced, aminated, deaminated, hydroxylated, dehydroxylated, hydrolyzed, dehydrolyzed, alkylated, dealkylated, acylated, deacylated, phosphorylated, dephosphorylated, thiophoshoramidated, dethiophoshoramidated, phoshoramidated or dephosphoramidated to produce the active compound. A compound of this invention possesses antiviral activity against a Flavivirus or is metabolized to a compound that exhibits such activity. The 2′-chloro-2′-fluoro nucleoside can also be administered as a 5′-phosphoether lipid, a bisphosphoramidate, a 3′,5′-cyclic phosphoramidate, a 3′,5′-cyclic thiophosphoramidate, a DTE conjugate, a mixed phosphoramidate-SATE derivative or a “SATE” derivative.

The term “phosphonic acid” refers to the group —P(O)(OH)₂.

The term “substituted” or “optionally substituted” indicates that the moiety can have at least one additional substituent selected from the group consisting of azido, cyano, halogen (fluoro, chloro, bromo, or iodo), alkyl, alkenyl, alkynyl, cycloalkyl, heterocycle, aryl, heteroaryl, haloalkyl (e.g. CHF₂, CH₂F, CF₃), hydroxyl, alkoxy, amino, —NH(C₁-C₆ unsubstituted alkyl), —NH(C₁-C₆ substituted alkyl), —NH—(C₀-C₂alkyl)(C₃-C₈cycloalkyl), —NH—(C₀-C₂alkyl)(C₃-C₈heterocycle), —NH—(C₀-C₂alkyl)(aryl), —N(C₁-C₆ unsubstituted alkyl)₂, —N(C₁-C₆ unsubstituted alkyl)(C₁-C₆ substituted alkyl), —N(C₁-C₆ substituted alkyl)₂, —NH—(C₀-C₂alkyl)(C₃-C₈cycloalkyl), —NH—(C₀-C₂alkyl)(C₃-C₈heterocycle), —NH—(C₀-C₂alkyl)(aryl), acyl, nitro, sulfonate, sulfate, phosphate, phosphonate, or thiol.

The term “sulfonate esters”, represented by the formula, R¹¹⁴S(O)₂OR¹¹⁵, comprise R¹¹⁴ wherein R¹¹⁴ is alkyl, haloalkyl, aralkyl or aryl. R¹¹⁵ is alkyl, aryl or aralkyl.

The term “sulfonic acid” refers to the group —SO₂OH.

The term “thiol” refers to the group —SH.

“Phosphate” refers to the group —OP(O)(OH)₂.

“Phosphate ester” refers to mono, di, and tri phosphates unless otherwise indicated.

The term “phosphoamidate”, “phosphoramidate”, or “phosphoroamidate” is a moiety that has a phosphorus bound to three oxygen groups and an amine (which may optionally be substituted). Suitable nonlimiting phosphoramidates useful in the present invention are described by Madela, Karolina and McGuigan in 2012, “Progress in the development of anti-hepatitis C virus nucleoside and nucleotide prodrugs”, Future Medicinal Chemistry 4(5), pages 625-650 10:1021/jm300074y and Dominique, McGuigan and Balzarini in 2004, “Aryloxy Phosphoramidate Triesters as Pro-Tides”, Mini Reviews in Medicinal Chemistry 4(4), pages 371-381. Additional phosphoramidates useful in the present invention are described in U.S. Pat. Nos. 5,233,031, 7,115,590, 7,547,704, 7,879,815, 7,888,330, 7,902,202, 7,951,789, 7,964,580, 8,071,568; 8,148,349, 8,263,575, 8,324,179, 8,334,270, 8,552,021, 8,563,530, 8,580,765, 8,735,372, 8,759,318; 6,455,513; and 8,334,270; as well as in European Patent Nos.: EP 2120565; and EP 1143995. Other phosphoramidates are described in the nucleoside patents described in the Background of the Invention.

Nonlimiting examples of a phosphoramidate include:

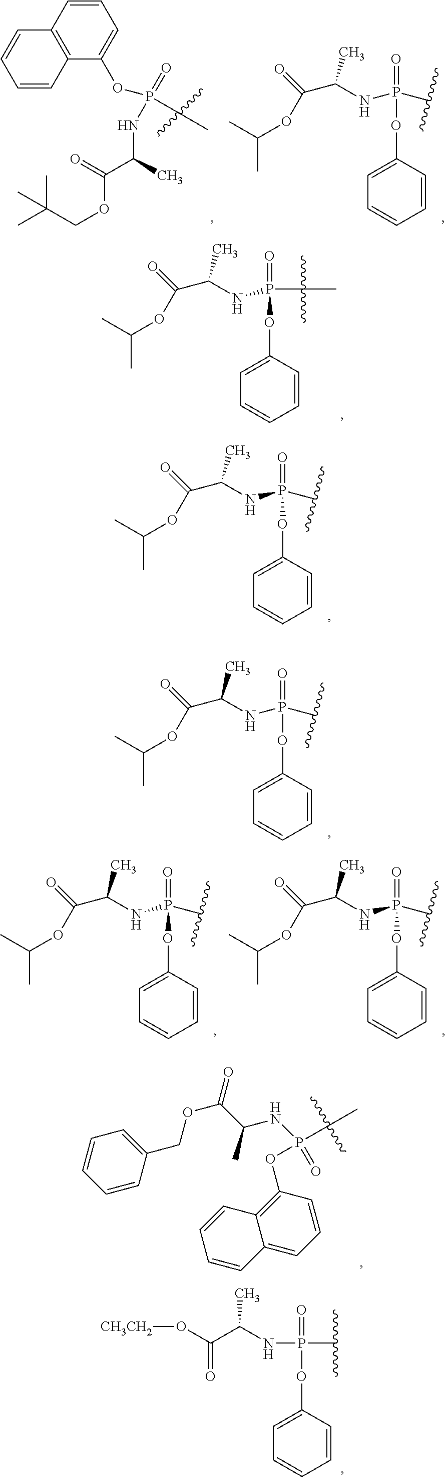

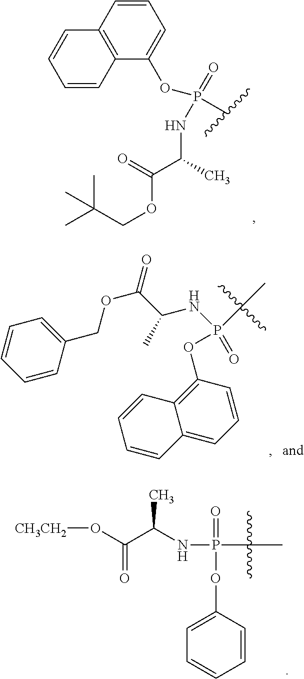

Other phosphoramidates included in the present invention are those of the structure:

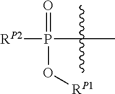

wherein:
R^{P1} is an optionally substituted linear, branched, or cyclic alkyl group, or an optionally substituted aryl, heteroaryl or heterocyclic group or a linked combination thereof; and
R^{P2} is a —NR^{N1}R^{N2} group or a B′ group;
wherein:
R^{N1} and R^{N2} are each independently H, C₁₋₈alkyl, (C₃-C₇cycloalkyl)C₀-C₄alkyl-, (aryl)C₀-C₄alkyl-, (C₃-C₆heterocyclo)C₀-C₄alkyl-, or (heteroaryl)C₀-C₄alky-; which may be optionally substituted; or
R^{N1} and R^{N2} along with the nitrogen atom to which that are attached, join to form a 3 to 7 membered heterocyclic ring;
B′ is a

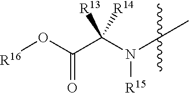

group;
wherein:
R¹³ is hydrogen, (C₁-C₈)alkyl, (C₂-C₅)alkenyl, (C₂-C₈)alkynyl, (C₃-C₈cycloalkyl)C₀-C₄alkyl-, (aryl)C₀-C₄alkyl-, (C₃-C₆heterocyclo)C₀-C₄alkyl-, (heteroaryl)C₀-C₄alky-, or the sidechain of an amino acid, for example a sidechain of an amino acid (as otherwise described herein) often selected from the group consisting of alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan, or tyrosine (often R¹³ is hydrogen, methyl, isopropyl, or isobutyl);
R¹⁴ is hydrogen, (C₁-C₅)alkyl, (C₂-C₅)alkenyl, (C₂-C₅)alkynyl, (C₃-C₈cycloalkyl)C₀-C₄alkyl-, (aryl)C₀-C₄alkyl-, (C₃-C₆heterocyclo)C₀-C₄alkyl-, (heteroaryl)C₀-C₄alky-, or the sidechain of an amino acid, for example a sidechain of an amino acid (as otherwise described herein) often selected from the group consisting of alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan, or tyrosine (often R¹⁴ is hydrogen, methyl, isopropyl, or isobutyl);
R¹⁵ is hydrogen or C₁-C₃alkyl; or
R¹³ and R¹⁴ can form a (C₃-C₇)cycloalkyl or (C₃-C₇)heterocyclic group; or
R¹³ and R¹⁴ or R¹⁶ can form (C₃-C₆)heterocyclic group; and
R¹⁶ is hydrogen, (C₁-C₆)alkyl, (C₃-C₆)alkenyl, (C₃-C₆)alkynyl, (C₃-C₈cycloalkyl)C₀-C₄alkyl, (aryl)C₀-C₄alkyl-, (C₃-C₆heterocyclo)C₀-C₄alkyl-, (heteroaryl)C₀-C₄alky-.
Preferred R^{P1} groups include optionally substituted phenyl, naphthyl, and monocyclic heteroaryl groups, especially those groups (particularly lipophilic groups) which enhance bioavailability of a compound in the cells of the patient, and which exhibit reduced toxicity, enhanced therapeutic index and enhanced pharmacokinetics (a compound is metabolized and excreted more slowly).

The term “thiophosphoamidate”, “thiophosphoramidate”, or “thiophosphoroamidate” is a moiety that has a phosphorus bound to sulfur, two oxygen groups and an amine (which may optionally be substituted). Nonlimiting examples of thiophosphoramidates included in the present invention are described in U.S. Pat. No. 8,772,474 and WO 2012/040124.

Thiophosphoramidate groups for use in the present invention include those of the structures:

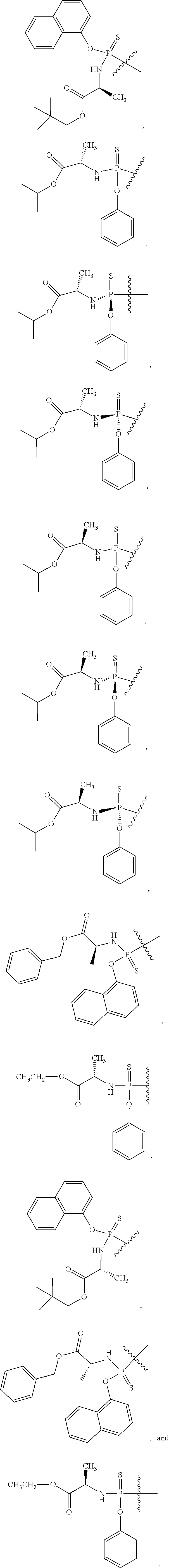

Other thiophosphoramidates included in the present invention are those of the structure:

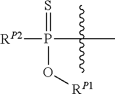

wherein:
R^{P1} is an optionally substituted linear, branched, or cyclic alkyl group, or an optionally substituted aryl, heteroaryl or heterocyclic group or a linked combination thereof; and
R^{P2} is a —NR^{N1}R^{N2} group or a B′ group;
wherein:
R^{N1} and R^{N2} are each independently H, C₁₋₈alkyl, (C₃-C₇cycloalkyl)C₀-C₄alkyl-, (aryl)C₀-C₄alkyl-, (C₃-C₆heterocyclo)C₀-C₄alkyl-, or (heteroaryl)C₀-C₄alky-; which may be optionally substituted; or
R^{N1} and R^{N2} along with the nitrogen atom to which that are attached, join to form a 3 to 7 membered heterocyclic ring;

B′ is a

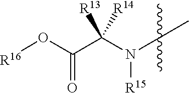

group;
wherein:
R¹³ is hydrogen, (C₁-C₈)alkyl, (C₂-C₈)alkenyl, (C₂-C₈)alkynyl, (C₃-C₈cycloalkyl)C₀-C₄alkyl-, (aryl)C₀-C₄alkyl-, (C₃-C₆heterocyclo)C₀-C₄alkyl-, (heteroaryl)C₀-C₄alky-, or the side chain of an amino acid, for example a side chain of an amino acid (as otherwise described herein) often selected from the group consisting of alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan, or tyrosine (often R¹³ is hydrogen, methyl, isopropyl, or isobutyl);
R¹⁴ is hydrogen, (C₁-C₅)alkyl, (C₂-C₈)alkenyl, (C₂-C₈)alkynyl, (C₃-C₈cycloalkyl)C₀-C₄alkyl-, (aryl)C₀-C₄alkyl-, (C₃-C₆heterocyclo)C₀-C₄alkyl-, (heteroaryl)C₀-C₄alky-, or the sidechain of an amino acid, for example a side chain of an amino acid (as otherwise described herein) often selected from the group consisting of alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan, or tyrosine (often R¹⁴ is hydrogen, methyl, isopropyl, or isobutyl);
R¹⁵ is hydrogen or C₁-C₃alkyl; or
R¹⁵ and one of R¹³ or R¹⁴ can form a (C₃-C₇)cycloalkyl or (C₃-C₇)heterocyclic group; and
   R¹⁶ is hydrogen, (C₁-C₆)alkyl, (C₃-C₆)alkenyl, (C₃-C₆)alkynyl, (C₃-C₈cycloalkyl)C₀-C₄alkyl, (aryl)C₀-C₄alkyl-, (C₃-C₆heterocyclo)C₀-C₄alkyl-, (heteroaryl)C₀-C₄alky-.

In certain nonlimiting embodiments, R^{P1} is selected from the group consisting of optionally substituted phenyl, naphthyl, and monocyclic heteroaryl groups.

The term “D-configuration” as used in the context of the present invention refers to the principle configuration which mimics the natural configuration of sugar moieties as opposed to the unnatural occurring nucleosides or “L” configuration. The term “B” or “β anomer” is used with reference to nucleoside analogs in which the nucleoside base is configured (disposed) above the plane of the furanose moiety in the nucleoside analog.

The term “host”, as used herein, refers to a unicellular or multicellular organism in which a Flavivirus can replicate, including cell lines and animals, and typically a human. The term host specifically refers to infected cells, cells transfected with all or part of an Flavivirus genome, and animals, in particular, primates (including chimpanzees) and humans. In most animal applications of the present invention, the host is a human patient. Veterinary applications, in certain indications, however, are clearly anticipated by the present invention (such as chimpanzees). The host can be for example, bovine, equine, avian, canine, feline, etc.

The term “Flavivirus” or “Flaviviruses,” as used herein, refers to viruses of the genus Flavivirus.

### Isotopic Substitution

The present invention includes administration of an effective amount of a compound with desired isotopic substitutions of atoms, at amounts above the natural abundance of the isotope, i.e., enriched. Isotopes are atoms having the same atomic number but different mass numbers, i.e., the same number of protons but a different number of neutrons. By way of general example and without limitation, isotopes of hydrogen, for example, deuterium (²H) and tritium (³H) may be used anywhere in described structures. Alternatively, or in addition, isotopes of carbon, e.g., ¹³C and ¹⁴C, may be used. A typical isotopic substitution is deuterium for hydrogen at one or more locations on the molecule to improve the performance of the drug. The deuterium can be bound in a location of bond breakage during metabolism (an α-deuterium kinetic isotope effect) or next to or near the site of bond breakage (a β-deuterium kinetic isotope effect). Achillion Pharmaceuticals, Inc. (WO/2014/169278 and WO/2014/169280) describes deuteration of nucleotides to improve their pharmacokinetics or pharmacodynamics, including at the 5-position of the molecule.

Substitution with isotopes such as deuterium can afford certain therapeutic advantages resulting from greater metabolic stability, such as, for example, increased in vivo half-life or reduced dosage requirements. Substitution of deuterium for hydrogen at a site of metabolic break down can reduce the rate of or eliminate the metabolism at that bond. At any position of the compound that a hydrogen atom may be present, the hydrogen atom can be any isotope of hydrogen, including protium (¹H), deuterium (²H) and tritium (³H). Thus, reference herein to a compound encompasses all potential isotopic forms unless the context clearly dictates otherwise.

The term “isotopically-labeled” analog refers to an analog that is a “deuterated analog”, a “¹³C-labeled analog,” or a “deuterated/¹³C-labeled analog.” The term “deuterated analog” means a compound described herein, whereby an H-isotope, i.e., hydrogen/protium (¹H), is substituted by an H-isotope, i.e., deuterium (²H). Deuterium substitution can be partial or complete. Partial deuterium substitution means that at least one hydrogen is substituted by at least one deuterium. In certain embodiments, the isotope is 90, 95, 96, 97, 98 or 99% or more enriched in an isotope at any location of interest. In some embodiments, it is deuterium that is 90, 95, 96, 97, 98 or 99% enriched at a desired location. Unless indicated to the contrary, the deuteration is at least 80% at the selected location. Deuteration of the nucleoside can occur at any replaceable hydrogen that provides the desired results.

### III. Methods of Treatment or Prophylaxis

Treatment, as used herein, refers to the administration of an effective amount of an active compound to a host who is infected with a Flavivirus, in particular dengue virus, yellow fever virus, Zika virus, or West Nile virus, and wherein the host is typically a human.

The term “prophylactic” or “preventative” when used herein refers to the administration of an effective amount of an active compound to prevent or reduce the likelihood of an occurrence of the viral disorder. The present invention includes both treatment and prophylactic or preventative therapies. In one embodiment, an effective amount of the active compound is administered to a host, typically a human, who has been exposed to and thus is at risk of infection by a Flavivirus infection.

The invention includes treatment of an infection caused by a Flavivirus, including drug resistant and multidrug resistant forms of a Flavivirus and related disease states, conditions, or complications of a Flavivirus infection, as well as other conditions that are secondary to a Flavivirus infection, such as weakness, loss of appetite, weight loss, breast enlargement (especially in men), rash (especially on the palms), difficulty with clotting of blood, spider-like blood vessels on the skin, confusion, coma (encephalopathy), buildup of fluid in the abdominal cavity (ascites), esophageal varices, portal hypertension, kidney failure, enlarged spleen, decrease in blood cells, anemia, thrombocytopenia, jaundice, and hepatocellular cancer, among others. The treatment comprises administering to a host in need thereof, typically a human, an effective amount of at least one 2′-chloro-2′-fluoro-N²-amino-N⁶-methylamino purine nucleotide phosphoramidate as described herein, optionally in combination with at least one additional bioactive agent, for example, an additional anti-Flavivirus agent, further in combination with a pharmaceutically acceptable carrier additive and/or excipient.

In one embodiment, an active compound is administered in an effective amount to a host, typically a human, that is infected with a Flavivirus.

In one embodiment, an active compound is administered in an effective amount to a host, typically a human, that is infected with Dengue virus.

In one embodiment, an active compound is administered in an effective amount to a host, typically a human, that is infected with Yellow Fever virus.

In one embodiment, an active compound is administered in an effective amount to a host, typically a human, that is infected with Zika virus.

In one embodiment, an active compound is administered in an effective amount to a host, typically a human, that is infected with West Nile virus.

In one embodiment, the Flavivirus infection is dengue fever. In a further embodiment, the dengue fever is Dengue virus type 1, type 2, type 3, or type 4. In one embodiment, the Flavivirus infection is West Nile fever. In one embodiment, the Flavivirus infection is Yellow Fever. In one embodiment, the Flavivirus infection is from the Zika virus.

### IV. Pharmaceutical Compositions

In an aspect of the invention, pharmaceutical compositions according to the present invention comprise an anti-Flavivirus effective amount of at least one of the 5′-stabilized 2′-chloro-2′-fluoro-N²-amino-N⁶-methylamino purine nucleotide compound, or a pharmaceutically acceptable salt thereof, described herein, optionally in combination with a pharmaceutically acceptable carrier, additive, or excipient, further optionally in combination or alternation with at least one other active compound. In certain embodiments, the treated virus is dengue virus. In certain embodiments, the treated virus is yellow fever virus. In certain embodiments, the treated virus is Zika virus. In certain embodiments, the treated virus is West Nile virus.

In an aspect of the invention, pharmaceutical compositions according to the present invention comprise an anti-Flavivirus effective amount of at least one of the active 2′-chloro-2′-fluoro-N²-amino-N⁶-methylamino purine nucleotide phosphoramidate compounds described herein, in combination with a pharmaceutically acceptable carrier, additive, or excipient, and further in combination with an effective amount of at least one other antiviral agent, such as an anti-Flavivirus agent.

One of ordinary skill in the art will recognize that a therapeutically effective amount will vary with the infection or condition to be treated, its severity, the treatment regimen to be employed, the pharmacokinetics of the agent used, as well as the patient or subject (animal or human) to be treated, and such therapeutic amount can be determined by the attending physician or specialist. In certain embodiments, the patient is a human.

A 2′-chloro-2′-fluoro-N²-amino-N⁶-methylamino purine nucleotide phosphoramidate used in the present invention can be formulated in an admixture with a pharmaceutically acceptable carrier. In general, it is typical to administer the pharmaceutical composition in orally administrable form, but certain formulations may be administered via a parenteral, intravenous, intramuscular, inhalation, topical, transdermal, buccal, subcutaneous, suppository, or other route, including intranasal spray. Intravenous and intramuscular formulations are often administered in sterile saline. One of ordinary skill in the art may modify the formulations to render them more soluble in water or other vehicle, for example, this can be easily accomplished by minor modifications (salt formulation, esterification, etc.) which are well within the ordinary skill in the art. It is also well within the routineers' skill to modify the route of administration and dosage regimen of a particular compound in order to manage the pharmacokinetics of a present compound for maximum beneficial effect in patients.

In certain pharmaceutical dosage forms, the prodrug form of a compound, especially including acylated (acetylated or other), and ether (alkyl and related) derivatives, phosphate esters, thiophosphoramidates, phosphoramidates, and various salt forms of a present compound, is typical. One of ordinary skill in the art will recognize how to readily modify a present compound to prodrug form to facilitate delivery of the active compound to a targeted site within the host organism or patient. The routineer also will take advantage of favorable pharmacokinetic parameters of the prodrug forms, where applicable, in delivering a present compound to a targeted site within the host organism or patient to maximize the intended effect of the compound.

The amount of compound included within therapeutically active formulations according to the present invention is an effective amount for treating the Flavivirus infection, reducing the likelihood of a Flavivirus infection or the inhibition, reduction, and/or abolition of a Flavivirus or its secondary effects, including disease states, conditions, and/or complications which occur secondary to a Flavivirus infection. In general, a therapeutically effective amount of the present compound in pharmaceutical dosage form usually ranges from about 0.001 mg/kg to about 100 mg/kg per day or more, more often, slightly less than about 0.1 mg/kg to more than about 25 mg/kg per day of the patient or considerably more, depending upon the compound used, the condition or infection treated and the route of administration. The active nucleoside compound according to the present invention is often administered in amounts ranging from about 0.1 mg/kg to about 15 mg/kg per day of the patient, depending upon the pharmacokinetics of the agent in the patient. This dosage range generally produces effective blood level concentrations of active compound which may range from about 0.001 to about 100, about 0.05 to about 100 micrograms/cc of blood in the patient.

Often, to treat, prevent or delay the onset of these infections and/or to reduce the likelihood of a Flavivirus infection, or a secondary disease state, condition or complication of a Flavivirus infection, the compositions will be administered in oral dosage forms in amounts ranging from about 100 milligrams up to about 1,200 mg or more at least once a day, for example, at least about 400, 450, 500, 550, 600, 650, 700, 750, 800, 850, 900, 950, 1,000, 1,050, 1,100, 1,150, or 1,200 milligrams, once, twice, three, or four times per day.

In certain embodiments, the 2′-chloro-2′-fluoro-N²-amino-N⁶-methylamino purine nucleotide phosphoramidate will be administered in oral dosage forms in amounts from about 500 milligrams to about 850 milligrams, for example at least about 500, 525, 550, 575, 600, 625, 650, 675, 700, 725, 750, 775, 800, 825, 850 milligrams or more at least once a day. In certain embodiments, the composition will be administered in an oral dosage form in an amount from about 500 mg to at least about 650 mg or more once, twice, three or four times per day. In certain embodiments, the composition will be administered in an oral dosage form in an amount from about 600 mg to at least about 750 mg or more once, twice, three or four times per day. In certain embodiments, the composition will be administered in an oral dosage form in an amount from about 650 mg to at least about 850 mg or more once, twice, three or four times per day. The compound is often administered orally, but may be administered parenterally, topically, or in suppository form, as well as intranasally, as a nasal spray or as otherwise described herein.

In certain embodiments, the 2′-chloro-2′-fluoro-N²-amino-N⁶-methylamino purine nucleotide phosphoramidate compound of the present invention is used as the hemisulfate salt. In certain embodiments, the hemisulfate salt of the compound is administered in oral dosage forms in amounts ranging from 400 milligrams up to about 1,200 milligrams, at least once per day, for example once, twice, three times or four times per day. In certain embodiments, at least about 400, 450, 500, 550, 600, 650, 700, 750, 800, 850, 900, 950, or 1,000 milligrams of the hemisulfate salt of the compound of the present invention is administered once, twice, three times or four times per day.

In certain embodiments, the hemisulfate salt of a 2′-chloro-2′-fluoro-N²-amino-N⁶-methylamino purine nucleotide phosphoramidate will be administered in oral dosage forms in amounts from about 500 milligrams to about 850 milligrams, for example about 500, 525, 550, 575, 600, 625, 650, 675, 700, 725, 750, 775, 800, 825, 850 milligrams or more at least once a day. In certain embodiments, from about 500 milligrams to at least about 650 milligrams of the hemisulfate salt of the compound is administered once, twice, three times or four times per day. In certain embodiments, from about 600 milligrams to at least about 750 milligrams of the hemisulfate salt of the compound is administered once, twice, three times or four times per day. In certain embodiments, from about 650 to at least about 850 milligrams of the hemisulfate salt of the compound is administered once, twice, three times or four times per day.

In the case of the co-administration of a 2′-chloro-2′-fluoro-N2-amino-N6-methylamino purine nucleotide phosphoramidate in combination with another anti-Flavivirus compound as otherwise described herein, the amount of the compound to be administered ranges from about 0.01 mg/kg of the patient to about 500 mg/kg or more of the patient or considerably more, depending upon the second agent to be co-administered and its potency against the virus, the condition of the patient and severity of the disease or infection to be treated and the route of administration. In certain embodiments, the 2′-chloro-2′-fluoro-N²-amino-N⁶-methylamino purine nucleotide phosphoramidate of the present invention is administered in an amount ranging from about 500 milligrams to about 850 milligrams once, twice, three, or four times per day when administered in combination with another anti-Flavivirus compound as described herein. The other anti-Flavivirus agent may for example be administered in amounts ranging from about 0.01 mg/kg to about 500 mg/kg. In certain embodiments, a compound may be often administered in an amount ranging from about 0.5 mg/kg to about 50 mg/kg or more (usually up to about 100 mg/kg), generally depending upon the pharmacokinetics of the two agents in the patient. These dosage ranges generally produce effective blood level concentrations of active compound in the patient.

For purposes of the present invention, a preventive or prophylactically effective amount of the compositions according to the present invention falls within the same concentration range as set forth above for therapeutically effective amount and is usually the same as a therapeutically effective amount.

Administration of an effective amount of an active compound may range from continuous intravenous drip to several oral or intranasal administrations per day (for example, Q.I.D.) or transdermal administration and may include oral, topical, parenteral, intramuscular, intravenous, sub-cutaneous, transdermal (which may include a penetration enhancement agent), buccal, and suppository administration, among other routes of administration. Enteric coated oral tablets may also be used to enhance bioavailability of a compound for an oral route of administration. The most effective dosage form will depend upon the bioavailability/pharmacokinetics of the particular agent chosen as well as the severity of disease in the patient. Oral dosage forms are particularly typical, because of ease of administration and prospective favorable patient compliance.

To prepare a pharmaceutical composition according to the present invention, a therapeutically effective amount of one or more of the compounds according to the present invention is often intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs, and solutions, suitable carriers and additives including, but not limited to, water, glycols, oils, alcohols, flavoring agents, preservatives, coloring agents may be used. For solid oral preparations such as powders, tablets, capsules, and for solid preparations such as suppositories, suitable carriers and additives including, but not limited to, starches, sugar carriers, such as dextrose, manifold, lactose, and related carriers, diluents, granulating agents, lubricants, binders, disintegrating agents may be used. If desired, the tablets or capsules may be enteric-coated or sustained release by standard techniques. The use of these dosage forms may significantly enhance the bioavailability of a compound in the patient.

For parenteral formulations, the carrier will usually comprise sterile water or aqueous sodium chloride solution, though other ingredients, including those which aid in dispersion, also may be included. Of course, where sterile water is to be used and maintained as sterile, the compositions and carriers must also be sterilized. Injectable suspensions may also be prepared, in which case appropriate liquid carriers, suspending agents, and the like may be employed.

Liposomal suspensions (including liposomes targeted to viral antigens) may also be prepared by conventional methods to produce pharmaceutically acceptable carriers. This may be appropriate for the delivery of free nucleosides, acyl/alkyl nucleosides or phosphate ester prodrug forms of a nucleoside compound according to the present invention.

In typical embodiments, according to the present invention, a compound and composition is used to treat, prevent or delay a Flavivirus infection or a secondary disease state, condition or complication of a Flavivirus infection.

### V. Combination and Alternation Therapy

It is well recognized that drug-resistant variants of viruses can emerge after prolonged treatment with an antiviral agent. Drug resistance most typically occurs by mutation of a gene that encodes for an enzyme used in viral replication. The efficacy of a drug against a Flavivirus infection can be prolonged, augmented, or restored by administering the compound in combination or alternation with another, and perhaps even two or three other, antiviral compounds that induce a different mutation or act through a different pathway, from that of the principal drug. Alternatively, the pharmacokinetics, bio distribution, half-life, or other parameter of the drug can be altered by such combination therapy (which may include alternation therapy if considered concerted). Since the disclosed 2′-chloro-2′-fluoro-N²-amino-N⁶-methylamino purine nucleotide phosphoramidate are polymerase inhibitors, it may be useful to administer an effective amount of a 2′-chloro-2′-fluoro-N²-amino-N⁶-methylamino purine nucleotide phosphoramidate to a host in need thereof in combination with an effective amount of, for example a:
(1) A protease inhibitor, such as an NS2B/NS3 protease inhibitor;
(2) A compound which disrupts the NS3/NS4B complex, such as but not limited to JNJ-A07;
(3) Another polymerase inhibitor;
(4) Interferon alfa-2a, which may be pegylated or otherwise modified, and/or ribavirin;
(5) Non-substrate-based inhibitor;
(6) Helicase inhibitor;
(7) Antisense oligodeoxynucleotide (S-ODN);
(8) Aptamer;
(9) Nuclease-resistant ribozyme;
(10) iRNA, including microRNA and siRNA;
(11) Antibody, partial antibody or domain antibody to the virus; or
(12) Viral antigen or partial antigen that induces a host antibody response.

### VI. Process of Preparation of 2′-Chloro-2′-Fluoro-N²-Amino-N⁶-Methylamino Purine Nucleotide Phosphoramidates of the Invention

General methods for providing a compound of the present invention are known in the art or described herein. The synthesis of 2′-chloro nucleotides is described in US 20150366888, WO 2014058801; WO 2015/066370 and WO 2015200219.

The following abbreviations are used in the synthetic schemes.
n-BuLi: n-Butyllithium
BSA: N,O-bis(trimethylsilyl)acetamide
CBr₄: Carbon tetrabromide
DBU: 1,8-Diazabicyclo[5.4.0]undec-7-ene
DAST: Diethylaminosulfur trifluoride
DCM: Dichloromethane
DIEA: N,N-Diisopropylethylamine
DMF: N,N-dimethylformamide
EA: Ethyl acetate
EtOAc: Ethyl acetate
EtOH: Ethanol
Et₃N: Triethylamine
Na₂SO₄: Sodium sulphate (anhydrous)
MeCN: Acetonitrile
MeNH₂: Methylamine
MeOH: Methanol
NaOH: Sodium hydroxide
Na₂SO₄: Sodium sulfate
Na₂S₂O₃: Sodium thiosulfate
NaHCO₃: Sodium bicarbonate
NH₄Cl: Ammonium chloride
NH₄OH: Ammonium hydroxide
NLT: Not less than
PE: Petroleum ether
Ph₃P: Triphenylphosphine
pTSA H₂O: p-Toluenesulfonic acid monohydrate
RT: Room Temperature
Silica gel (230 to 400 mesh, Sorbent)
TBAF: Tetrabutylammonium fluoride
THF: Tetrahydrofuran (THF), anhydrous
TMSCI: Chlorotrimethylsilane
TMSOTf: Trimethylsilyl trifluoromethanesulfonate
TIPDSiCl₂: 1,3-Dichloro-1,1,3,3-tetraisopropyldisiloxane
t-BuMgCl: t-Butyl magnesium chloride
t-BuOK: Sodium tert-butoxide
t-BuOH: Tert-butanol

### EXAMPLES

### General Methods

¹H, ¹⁹F and ³¹P NMR spectra were recorded on a 300 MHz Fourier transform Brücker spectrometer. Spectra were obtained from samples prepared in 5 mm diameter tubes in CDCl₃, CD₃OD or DMSO-d₆. The spin multiplicities are indicated by the symbols s (singlet), d (doublet), t (triplet), m (multiplet) and br (broad). Coupling constants (J) are reported in Hz. MS spectra were obtained using electrospray ionization (ESI) on an Agilent Technologies 6120 quadrupole MS apparatus. The reactions were generally carried out under a dry nitrogen atmosphere using Sigma-Aldrich anhydrous solvents. All common chemicals were purchased from commercial sources.

NMR spectra of compounds 1-11 were recorded on a 400 MHz Fourier transform Brücker spectrometer. Spectra were obtained from samples prepared in 5 mm diameter tubes in CDCl₃, CD₃OD or DMSO-d6. The spin multiplicities are indicated by the symbols s (singlet), d (doublet), t (triplet), m (multiplet) and, br (broad). Coupling constants (J) are reported in Hz. MS spectra were obtained using electrospray ionization (ESI) on an Agilent Technologies 6120 quadrupole MS apparatus. The reactions were generally carried out under a dry nitrogen atmosphere using Sigma-Aldrich anhydrous solvents. All common chemicals were purchased from commercial sources.

### Preparation of Stereospecific Phosphorus Enantiomers

Certain active compounds described herein have a chiral phosphorus moiety. Any of active compound described herein can be provided as an isolated phosphorus enantiomeric form, for example, at least 80%, 90%, 95%, 96%, 97% or 98% of the R or S enantiomer, using methods known to those of skill in the art. For example, there are a number of publications that describe how to obtain such compounds including, but not limited to, column chromatography, for example, as described in U.S. Pat. Nos. 8,859,756; 8,642,756 and 8,333,309 to Ross, et al. PPAL-RS can be separated by supercritical fluid chromatography into PPAL-R and PPAL-S, as described in Ross et al. J. Org. Chem. 2011, 76, 8311.

### Example 1. Modification of the 2-Amino Moiety in an Active Compound

One of ordinary skill in the art can add a substituent to the 2-amino purine moiety by methods well known to those skilled in the art. One non-limiting process is provided here, and others can be easily adapted. ((2R,3R,4R,5R)-3-(benzoyloxy)-5-bromo-4-fluoro-4-methyltetrahydrofuran-2-yl)methyl benzoate, is treated with commercially available 2,6-dichloropurine, a base and a mixture of organic solvents at an elevated temperature to generate (2R,3R,4R,5R)-5-(2,6-dichloro-9H-purin-9-yl)-2-(benzoyloxymethyl)-4-fluoro-4-methyl-tetrahydrofuran-3-yl benzoate. In one embodiment, the base is potassium tert-butoxide. In one embodiment, the mixture of organic solvents comprises tert-butanol and acetonitrile. The compound, (2R,3R,4R,5R)-5-(2,6-dichloro-9H-purin-9-yl)-2-(benzoyloxymethyl)-4-fluoro-4-methyltetrahydrofuran-3-yl benzoate is treated with an amine, a base and an organic solvent at ambient temperature to generate 2-chloro-N⁶-substituted purines. In one embodiment, the amine is methylamine. In one embodiment, the base is triethylamine. In one embodiment, the organic solvent is ethanol. One skilled in the art will also recognize that upon treatment with an amine and base, the benzoate groups on the nucleoside will simultaneously be removed to generate the deprotected furanose moiety. 2-Chloro-N⁶-substituted purines can then be treated with an amine, and an organic solvent in a sealed tube at an elevated temperature of about 100° C. to generate N², N⁶-disubstituted purine nucleosides of the present invention. In one embodiment, the amine is methylamine. In one embodiment, the organic solvent is ethanol. N², N⁶-Disubstituted purine nucleosides of the present invention can be treated with a base, isopropyl ((R,S)-(pentafluorophenoxy)-phenoxy-phosphoryl)-L-alaninate and an organic solvent at a reduced temperature to generate a compound of Formula I or Formula II. In one embodiment, the base is tert-butyl magnesium chloride. In one embodiment, the organic solvent is tetrahydrofuran.

### Example 2a. Preparation of PPAL-S

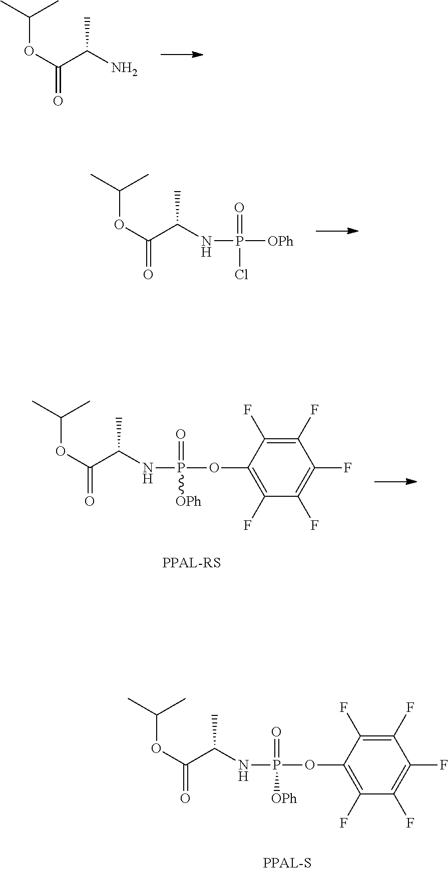

### Step 1. Preparation of Racemic PPAL

To a stirred solution of phenyl dichlorophosphate (250 g) in EtOAc (800 mL) was added isopropyl L-alaninate (200 g) in triethylamine (120 g) at −10° C. The reaction was stirred at −10° C. for 1 h. 2,3,4,5,6-Pentafluorophenol (220 g) in triethylamine (120 g) and EtOAc (400 mL) was added at −5° C. and stirred at −5° C. for 0.5 h. The reaction mixture was allowed to warm to 25° C. and stirred at 25° C. for 2 h. The solution was filtrated, washed with EtOAc (2×200 mL), and the combined organic phases were evaporated under vacuum to afford solid PPAL-RS (racemate).

### Step 2. Preparation of PPAL-S

To a stirred solution of PPAL-RS in EtOAc (200 mL) and n-heptane (1.4 L), was added 2,3,4,5,6-pentafluorophenol (10.1 g) in triethylamine (6 g), and the reaction was stirred for about 4-8 h. After the R-isomer of the solid was less than 0.5% of the reaction mixture, the solid was filtered. The solid was dissolved in EtOAc (4 L), washed with water (2×100 mL), brine (1 L), dried over anhydrous Na₂SO₄, and filtered. The solvent was removed under vacuum to afford PPAL-S (350 g).

¹H NMR (400 MHZ, DMSO-d₆) δ=7.42-7.40 (m, 2H), 7.24-7.22 (m, 3H), 6.87 (dd, J=14.1, 9.9 Hz, 1H), 4.90-4.84 (m, 1H), 3.94-3.88 (m, 1H), 1.27 (dd, J=7.1, 1.1 Hz, 3H), 1.15 (dd, J=6.2, 1.2 Hz, 6H) ppm. ¹³P NMR (160 MHz, DMSO-d6) δ=0.37 ppm.

### Example 2b. Preparation of PPAD-S

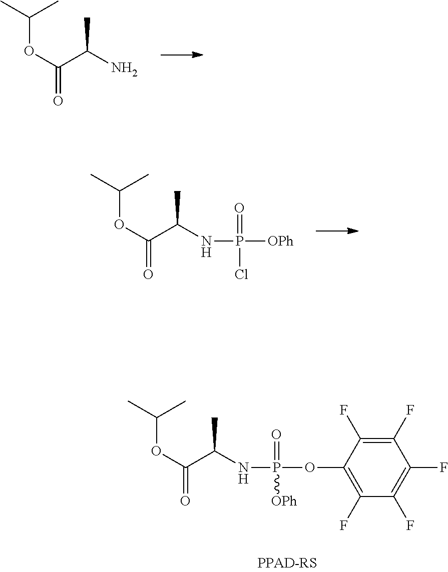

### Step 1. Preparation of Racemic PPAD (PPAD-RS)

Isopropyl-D-alaninate is added to a stirred solution of phenyldichlorophosphate in EtOAc. The reaction is stirred at −10° C. for one hour. Next, 2,3,4,5,6-pentafluorophenol in triethylamine is added at −5° C. and stirred at −5° C. for 0.5 h. The reaction mixture is warmed to 25° C. and stirred at 25° C. for 2 h. The solution is filtered, washed with EtOAc, and the combined organic phases are evaporated under vacuum to afford isopropyl ((perfluorophenoxy)(phenoxy)phosphoryl)-D-alaninate (PPAD-RS) as a racemate.

### Step 2. Preparation of PPAD-R and PPAD-S

Isopropyl ((perfluorophenoxy)(phenoxy)phosphoryl)-D-alaninate (PPAD-RS) is purified by supercritical fluid chromatography using a chiral stationary phase to provide PPAD-R and PPAD-S.

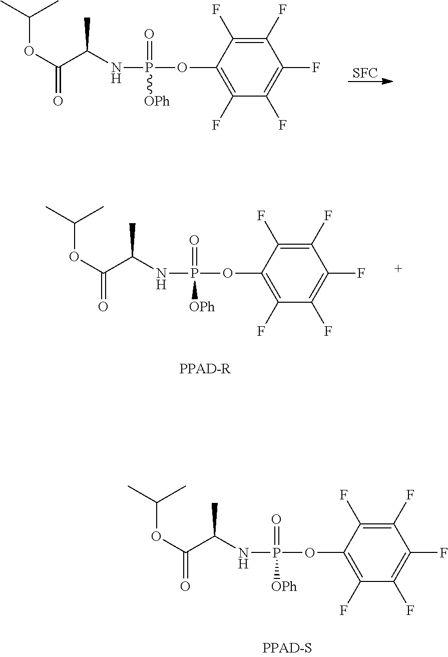

Example 3. Preparation of (S)-Isopropyl-2-(((S)-(((2R,3R,4S,5R)-5-(2-amino-6-(methylamino)-9H-purin-9-yl)-4-chloro-4-fluoro-3-hydroxytetrahydrofuran-2-yl)methoxy)(phenoxy)phosphoryl)amino) propanoate (8)

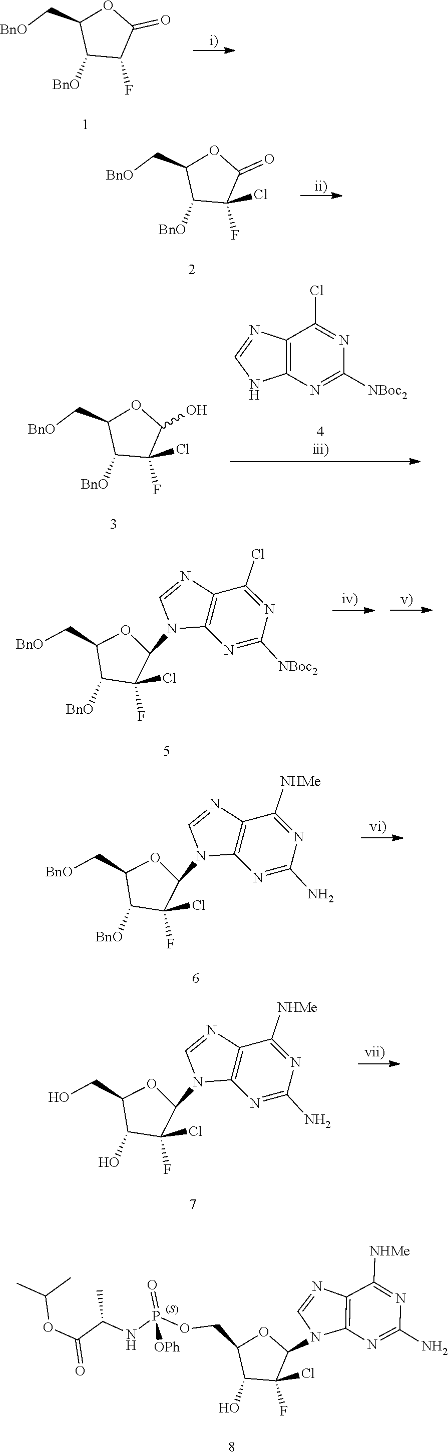

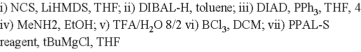

### Step 1.

### (3S,4R,5R)-4-(Benzyloxy)-5-((benzyloxy)methyl)-3-chloro-3-fluorodihydrofuran-2(3H)-one (2)

Commercially available compound 1 (4.0 g, 12.1 mmol, 1.0 equiv.) and NCS (2.4 g, 18.2 mmol, 1.5 equiv.) were dissolved in THF (60 mL) under N₂ atmosphere and cooled to −78° C. LiHMDS (24 mL, 24.0 mmol, 2 equiv.) was added dropwise over 20 minutes and the reaction mixture was stirred at −78° C. for 1 h. The reaction mixture was quenched with NH₄Cl aq. sat. solution (100 mL), the layers were separated, and the aqueous layer extracted with EtOAc (2×100 mL). The combined organic phases were dried over Na₂SO₄, filtered and concentrated. The crude mixture was purified by column chromatography (Pet. Ether/EtOAc, gradient from 100:0 to 80:20 to 0:100) to give product 2 (1.35 g, 31% yield) as a glassy solid.

### Step 2.

### (3S,4R,5R)-4-(Benzyloxy)-5-((benzyloxy)methyl)-3-chloro-3-fluorotetrahydrofuran-2-ol (3)

Compound 2 (1.35 g, 3.7 mmol, 1.0 equiv.) was dissolved in dry toluene (40 mL) under N₂ atmosphere and cooled to −78° C. DIBALH (5.5 mL, 5.5 mmol, 1.5 equiv.) was added dropwise and the reaction mixture was stirred at −78° C. for 45 minutes. The reaction mixture was quenched with MeOH at −78° C. then warmed up to RT. Rochelle's salt aq. sat. solution (1000 mL) was added, and the reaction mixture was stirred for 30 minutes then extracted with EtOAc (2×200 mL). The combined organic phases were dried over Na₂SO₄, filtered and concentrated. The crude mixture was purified by column chromatography (Pet. Ether/EtOAc, gradient from 100:0 to 0:100) to give product 3 (1.15 g, 85% yield) as a colorless oil.

### Step 3.

### Imidodicarbonic acid, (9-((2R,3S,4R,5R)-4-(benzyloxy)-5-((benzyloxy)methyl)-3-chloro-3-fluorotetrahydrofuran-2-yl)-6-chloro-9H-purin-2-yl)-, 1,3-bis(1,1-dimethylethyl) ester (5)

Compound 3 (1.15 g, 3.1 mmol, 1 equiv.) was dissolved in THF under N₂ atmosphere, protected base 4 (1.74 g, 4.7 mmol, 1.5 equiv.) and PPh₃ (987 mg, 3.8 mmol, 1.2 equiv.) were added at RT. At 0° C., DIAD (864 μL, 4.4 mmol, 1.4 equiv.) was added dropwise. The reaction mixture was stirred at RT for 1 h. Silica gel was added to the reaction mixture which was then concentrated. Residue was purified by column chromatography (Pet. Ether/EtOAc, gradient from 100:0 to 0:100) to give a mixture of alpha and beta anomers (1.7 g, 75% yield) as a pale-yellow solid which were separated after a second purification by column chromatography (Pet. Ether/EtOAc, gradient from 100:0 to 0:100) to give pure beta anomer 5 (720 mg, 32% yield) and pure alpha anomer (420 mg, 19% yield).

### Steps 4 and 5

### 9-((2R,3S,4R,5R)-4-(benzyloxy)-5-((benzyloxy)methyl)-3-chloro-3-fluorotetrahydrofuran-2-yl)-N⁶-methyl-9H-purine-2,6-diamine (6)

Compound 5 (470 mg, 0.65 mmol, 1 equiv.) was dissolved in a solution of MeNH₂ (33% in EtOH, 5 mL) and stirred in a sealed tube for 1 h at 80° C. The reaction mixture was evaporated to dryness and treated with a solution TFA/H₂O (4 mL/2 mL) at RT for 2 h then concentrated. The residue was purified by column chromatography (Pet. Ether/EtOAc, gradient from 100:0 to 0:100) to give product 6 (310 mg, 92% yield) as a white solid.

### Step 6.

### (2R,3R,5R)-5-(2-Amino-6-(methylamino)-9H-purin-9-yl)-4-chloro-4-fluoro-2-(hydroxymethyl)tetrahydrofuran-3-ol (7)

To a solution of compound 6 (271 mg, 0.53 mmol, 1.0 equiv.) in dry DCM (10 mL) under N₂ atmosphere at −80° ° C. was added boron trichloride (1 M in DCM, 2.1 mL, 2.11 mmol, 4.0 equiv.) dropwise and the reaction mixture was stirred from −80° C. to −30° C. for 1 h. The reaction mixture was cooled to −80° C. again and ammonia (2 M in MeOH, 2.11 mL, 4.22 mmol, 8 equiv.) was added. The reaction mixture was allowed to warm up to RT under N₂ atmosphere and directly loaded onto a pre-column filled with silica gel for purification by flash column chromatography (silica gel, DCM/MeOH, gradient from 100:0 to 0:100). After a second purification by reverse phase C18 chromatography (MeOH/H₂O gradient from 0:100 to 100:0) product 7 was obtained as a white solid (140 mg, 79% yield). ¹H NMR (400 MHZ, CD₃OD): δ 8.04 (s, 1H), 6.29 (d, J=14.8 Hz, 1H), 4.56 (dd, J=19.2 Hz, 8.8 Hz, 1H), 4.04-4.01 (m, 2H), 3.87 (dd, J=13.2, 3.3 Hz, 1H), 3.03 (s, 3H). MS (ESI) m/z calcd. for C₁₁H₁₅ClFN₆O₃[M+H]⁺333.7; found 333.7.

### Step 7.

### (S)-Isopropyl-2-(((S)-(((2R,3R,4S,5R)-5-(2-amino-6-(methylamino)-9H-purin-9-yl)-4-chloro-4-fluoro-3-hydroxytetrahydrofuran-2-yl)methoxy)(phenoxy)phosphoryl)amino) propanoate (8)

To a solution of compound 7 (56 mg, 0.168 mmol, 1 equiv.) in dry DMF (3 mL) under N₂ atmosphere was added tert-butylmagnesium chloride (1 M in N-methyl THF) (340 μL, 0.337 mmol, 2 equiv.) dropwise at −10° ° C. The solution was stirred for 20 minutes at 0° C. and for 40 minutes at RT. Then, the reaction mixture was cooled down to −10° C. and a solution of isopropyl ((S)-(pentafluorophenoxy)-phenoxy-phosphoryl)-L-alaninate (91 mg, 0.202 mmol, 1.2 equiv.) in dry DMF (1.0 mL) was added dropwise. The reaction was stirred from 0° ° C. to RT overnight then MeOH was added to quench the reaction and the reaction mixture was concentrated. The residue was purified by 2 consecutive column chromatographies (silica gel, Pet. Ether/EtOAc gradient from 90:10 to 0:100 then DCM/MeOH 100:0 to 90:10) and product 8 (11.2 mg, 11% yield) was obtained as a white solid. ¹H NMR (400 MHZ, CD₃OD) δ 7.82 (s, 1H), 7.36-7.16 (m, 5H), 6.30 (d, J=15.3 Hz, 1H), 4.87 (m, 2H, overlapped with H₂O), 4.53-4.51 (m, 2H), 4.19-4.17 (m, 1H), 3.92-3.87 (m, 1H), 3.04 (br. s, 3H), 1.30 (dd, J=7.11, 0.74 Hz, 3H), 1.18-1.15 (m, 6H). ³¹P NMR (121 MHZ, CD₃OD) δ 3.76 (s). MS (ESI) m/z calcd. for C₂₃H₃₁ClFN₇O₇P [M+H]+ 602.9; found 603.0.

### Example 4. Preparation of 2-Amino-9-((2R,3S,4R,5R)-3-chloro-3-fluoro-4-hydroxy-5-(hydroxymethyl) tetrahydrofuran-2-yl)-1H-purin-6(9H)-one (10)

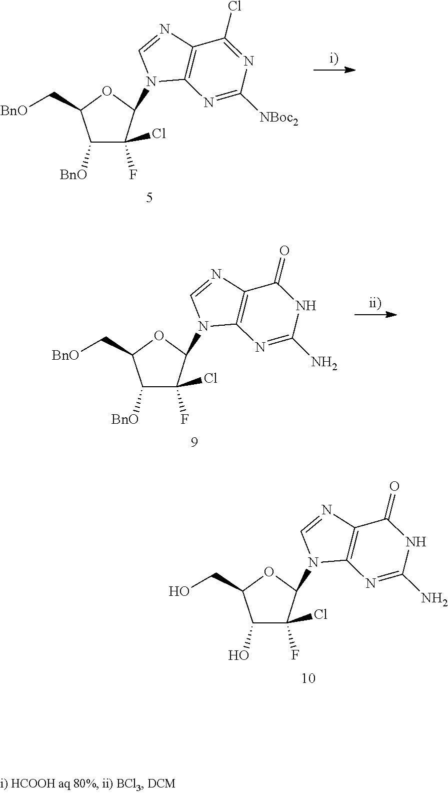

### Step 1.

### 2-Amino-9-((2R,3S,4R,5R)-4-(benzyloxy)-5-((benzyloxy)methyl)-3-chloro-3-fluorotetrahydrofuran-2-yl)-1H-purin-6(9H)-one (9)

Compound 5 (371 mg, 0.52 mmol, 1 equiv) was dissolved in a solution of aqueous formic acid (80%, 5 mL). The reaction mixture was stirred at 60° C. overnight then evaporated to dryness and co-evaporated with toluene. The residue was purified by column chromatography (DCM/MeOH, gradient from 100:0 to 9:1) to give product 9 as a white solid (190 mg, 74% yield).

### Step 2.

### 2-Amino-9-((2R,3S,4R,5R)-3-chloro-3-fluoro-4-hydroxy-5-(hydroxymethyl) tetrahydrofuran-2-yl)-1H-purin-6(9H)-one (10)

To a solution of compound 9 (190 mg, 0.38 mmol, 1.0 equiv.) in dry DCM (10 mL) under N₂ atmosphere at −80° C. was added boron trichloride (1 M in DCM, 0.15 mL, 1.52 mmol, 4.0 equiv.) dropwise and the reaction mixture was stirred from −80° C. to −30° C. for 1 h. The reaction mixture was cooled to −80° C. again and ammonia (2 M in MeOH, 1.5 mL, 3.04 mmol, 8 equiv.) was added. The reaction mixture was allowed to warm up to RT under N₂ atmosphere and directly loaded onto a pre-column filled with silica gel for purification by flash column chromatography (silica gel, DCM/MeOH, gradient from 100:0 to 0:100). After a second purification by reverse phase C18 chromatography (MeOH/H₂O gradient from 10:90 to 100:0) product 11 was obtained as a white solid (94 mg, 77% yield). ¹H NMR (400 MHZ, CD₃OD): δ 8.10 (s, 1H), 6.29 (d, J=14.8 Hz, 1H), 4.65 (dd, J=19.2 Hz, 8.9 Hz, 1H), 4.03-3.98 (m, 2H), 3.87-3.83 (m, 1H). MS (ESI) m/z calcd. for C₁₀H₁₂ClFN₅O₄ [M+H]⁺320.7; found 320.0.

### Example 5. Antiviral Activity of Compound 8 in Cells Infected with Various Flaviviruses

Huh-7 (human liver carcinoma; AcceGen Biotechnology, Fairfield, NJ) cells were maintained in Dulbecco's Modified Eagle medium (DMEM) supplemented with 10% fetal bovine serum (FBS) and 100 μg/mL penicillin and 100 μg/mL streptomycin (Lonza, Walkersville, MD). Cell cultures were maintained at 37° C. in an atmosphere of 5% CO₂ and >95% humidity. Infections were performed in EMEM supplemented with 5% FBS and 50 μg/mL gentamicin. Dengue virus (DENV-2) were obtained from ATCC (Manassas, VA). West Nile (WN02 Kern 515) and Yellow Fever (YFV 17D) were obtained from the University of Texas Medical Branch (Galveston, TX).

Test compounds were dissolved in DMSO at a concentration of 10 mM and serially diluted using eight half-log dilutions so that the highest test concentration was 100 μM). Each dilution was added to 5 wells of a 96-well plate with 80-100% confluent Huh-7 cells. Three wells of each dilution were infected with virus, and two wells remained uninfected as toxicity controls. Six untreated wells were infected as virus controls and six untreated wells were left uninfected to use as virus controls. Viruses were diluted to achieve MOIs of approximately 0.001 CCID₅₀ (50% cell culture infectious dose) per cell. Plates were incubated at 37° C. in a humidified atmosphere containing 5% CO₂. On day 5 (YFV) or day 6 (WNV and DENV-2) post-infection, when untreated virus control wells reached maximum cytopathic effect (CPE), the plates were stained with neutral red dye for approximately 2 hours (±15 minutes). Supernatant dye was removed, wells were rinsed with PBS, and the incorporated dye was extracted in 50:50 Sorensen citrate buffer/ethanol for >30 minutes. The optical density was read on a spectrophotometer at 540 nm and converted to percent of controls. The concentrations of test compound required to prevent virus-induced CPE by 50% (EC₅₀) and to cause 50% cell death in the absence of virus (CC₅₀) were calculated. Results are shown in Table 1.

TABLE 1
Results of Antiviral Activity Assay with Compound 8

| | | | EC₅₀ | CC₅₀ |
|---|---|---|---|---|
| Virus | Strain | Host Cell | (μM) | (μM) |
| Dengue-2 | New Guinea C | Huh-7 | 0.32 | >100 |
| West Nile | Kern 515, WNo2 | Huh-7 | 0.32 | >100 |
| Yellow Fever | YFV 17D | Huh-7 | 0.12 | >100 |

This specification has been described with reference to embodiments of the invention. Given the teaching herein, one of ordinary skill in the art will be able to modify the invention for a desired purpose and such variations are considered within the scope of the invention.

We claim:

1. A method comprising administering an effective amount of a compound of Formula I: 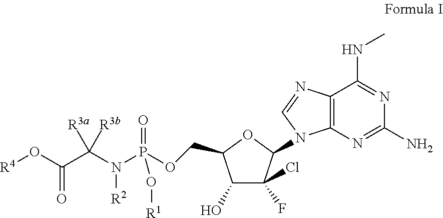
or a pharmaceutically acceptable salt thereof;
to treat a human host in need thereof infected with a Flavivirus;
wherein:
R¹ is C₁₋₆alkyl, C₃₋₇cycloalkyl, aryl, —(C₁-C₄alkyl)aryl, heteroaryl, or heteroalkyl;
R² is hydrogen or C₁₋₆alkyl;
R^{3a} and R^{3b} are independently selected from the group consisting of hydrogen, C₁₋₆alkyl, and C₃₋₇cycloalkyl; and
R⁴ is C₁₋₆alkyl, C₁₋₆haloalkyl, C₃₋₇cycloalkyl, —(C₁-C₄alkyl)aryl, aryl, heteroaryl, or heteroalkyl.

2. The method of claim 1, wherein R¹ is phenyl.

3. The method of claim 1, wherein R² is hydrogen.

4. The method of claim 1, wherein R^{3a} and R^{3b} are hydrogen and C₁₋₆alkyl.

5. The method of claim 1 wherein R⁴ is C₁₋₆alkyl.

6. The method of claim 1 wherein the compound is of formula: 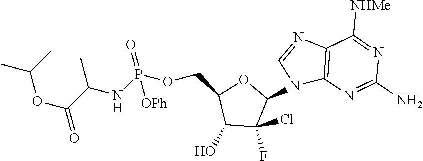 [SMILES: CNc1nc(N)nc2c1ncn2[C@@H]1O[C@H](COP(=O)(NC(C)C(=O)OC(C)C)Oc2ccccc2)[C@@H](O)[C@]1(F)Cl]
or a pharmaceutically acceptable salt thereof.

7. The method of claim 1 wherein the compound is selected from: 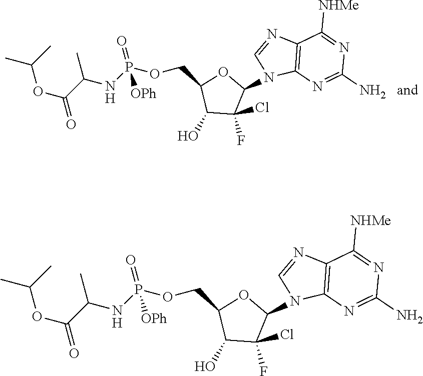 [SMILES: CNc1nc(N)nc2c1ncn2[C@@H]1O[C@H](CO[P@@](=O)(NC(C)C(=O)OC(C)C)Oc2ccccc2)[C@@H](O)[C@]1(F)Cl ; "and" ; CNc1nc(N)nc2c1ncn2[C@@H]1O[C@H](CO[P@](=O)(NC(C)C(=O)OC(C)C)Oc2ccccc2)[C@@H](O)[C@]1(F)Cl]
or a pharmaceutically acceptable salt thereof.

8. The method of claim 1 wherein the compound is: 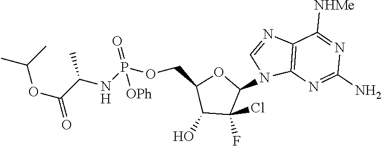 [SMILES: CNc1nc(N)nc2c1ncn2[C@@H]1O[C@H](COP(=O)(N[C@@H](C)C(=O)OC(C)C)Oc2ccccc2)[C@@H](O)[C@]1(F)Cl]
or a pharmaceutically acceptable salt thereof.

9. The method of claim 1 wherein the compound is: 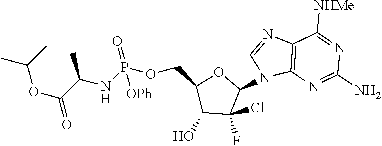 [SMILES: CNc1nc(N)nc2c1ncn2[C@@H]1O[C@H](COP(=O)(N[C@H](C)C(=O)OC(C)C)Oc2ccccc2)[C@@H](O)[C@]1(F)Cl]
or a pharmaceutically acceptable salt thereof.

10. The method of claim 1 wherein the compound is: 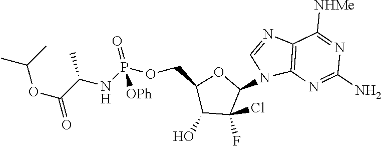 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@]1(F)Cl]
or a pharmaceutically acceptable salt thereof.

11. The method of claim 1, wherein the compound is: 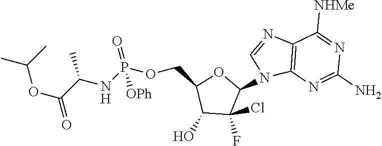 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@]1(F)Cl]
or a pharmaceutically acceptable salt thereof.

12. The method of claim 1 wherein the compound is: 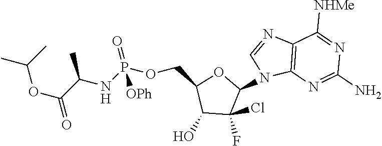 [SMILES: CNc1nc(N)nc2c1ncn2[C@@H]1O[C@H](CO[P@@](=O)(N[C@H](C)C(=O)OC(C)C)Oc2ccccc2)[C@@H](O)[C@]1(F)Cl]
or a pharmaceutically acceptable salt thereof.

13. The method of claim 1, wherein the compound is: 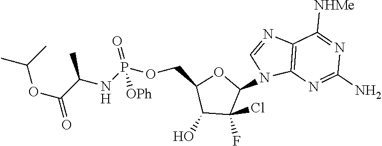 [SMILES: CNc1nc(N)nc2c1ncn2[C@@H]1O[C@H](CO[P@](=O)(N[C@H](C)C(=O)OC(C)C)Oc2ccccc2)[C@@H](O)[C@]1(F)Cl]
or a pharmaceutically acceptable salt thereof.

14. The method of claim 1 wherein the compound is:

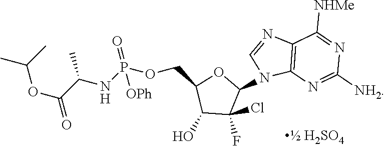 [SMILES: CNc1nc(N)nc2c1ncn2[C@@H]1O[C@H](COP(=O)(N[C@@H](C)C(=O)OC(C)C)Oc2ccccc2)[C@@H](O)[C@]1(F)Cl]

15. The method of claim 1 wherein the compound is:

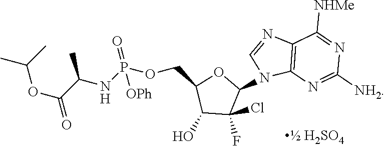 [SMILES: CNc1nc(N)nc2c1ncn2[C@@H]1O[C@H](COP(=O)(N[C@H](C)C(=O)OC(C)C)Oc2ccccc2)[C@@H](O)[C@]1(F)Cl]

16. The method of claim 1 wherein the compound is:

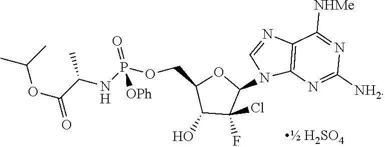 [SMILES: CNc1nc(N)nc2c1ncn2[C@@H]1O[C@H](CO[P@@](=O)(N[C@@H](C)C(=O)OC(C)C)Oc2ccccc2)[C@@H](O)[C@]1(F)Cl]

17. The method of claim 1, wherein the compound is:

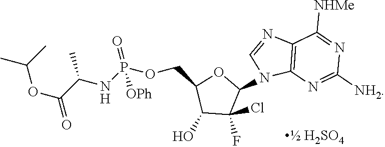 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@]1(F)Cl]

18. The method of claim 1 wherein the compound is:

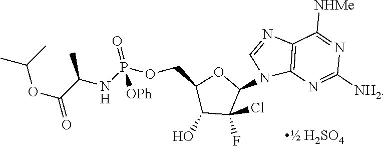 [SMILES: CNc1nc(N)nc2c1ncn2[C@@H]1O[C@H](CO[P@@](=O)(N[C@H](C)C(=O)OC(C)C)Oc2ccccc2)[C@@H](O)[C@]1(F)Cl]

19. The method of claim 1, wherein the compound is:

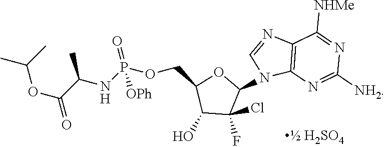 [SMILES: CNc1nc(N)nc2c1ncn2[C@@H]1O[C@H](CO[P@](=O)(N[C@H](C)C(=O)OC(C)C)Oc2ccccc2)[C@@H](O)[C@]1(F)Cl]

20. The method of claim 1, wherein the virus is dengue virus.

21. The method of claim 1, wherein the virus is yellow fever virus.

22. The method of claim 1, wherein the virus is West Nile virus.

23. The method of claim 1, wherein the virus is Zika virus.
